(19) United States
(12) Patent Application Publication
(10) Pub. No.: US 2026/0151163 A1
(43) Pub. Date: Jun. 4, 2026

(54) BONE ANCHOR ASSEMBLIES WITH TWO-PIECE RECEIVERS, SEPARATE RETAINING STRUCTURES, AND BOTTOM-LOADED SHANK HEADS

(71) Applicant: Roger P. Jackson, Prairie Village, KS (US)

(72) Inventors: Roger P. Jackson, Prairie Village, KS (US); James L. Surber, Kansas City, KS (US); Nathaniel D. Ginzton, Boise, ID (US)

(21) Appl. No.: 19/404,525

(22) Filed: Dec. 1, 2025

Related U.S. Application Data
(60) Provisional application No. 63/726,603, filed on Dec. 1, 2024.

(51) Int. Cl. A61B 17/70; A61B 17/00

(52) U.S. Cl. CPC A61B 17/7035; A61B 2017/00526

(57) ABSTRACT

A pivotal bone anchor assembly includes a two-piece receiver comprising an upper body having an open channel for receiving a rod and an annular lower end with a downwardly-extending first connection structure, and a cylindrical base having a spherical seating surface adjacent a bottom opening and an upwardly-extending second connection structure configured for coupling with the first connection structure to secure the cylindrical base to the upper body. The assembly also includes a bone anchor comprising a capture portion and an anchor portion, and a retaining structure configured to secure the capture portion within the cylindrical base. The assembly further includes a pressure insert having an upper surface configured to receive the rod and a lower surface configured to engage an upper surface of the capture portion. The retaining structure is positionable into the cylindrical base and the pressure insert is positionable into the upper body prior to the cylindrical base and the upper body being coupled together to complete the two-piece receiver.

### CROSS-REFERENCE TO RELATED APPLICATIONS

This application claims the benefit of U.S. Provisional Application No. 63/726,603, filed Dec. 1, 2024, which is incorporated by reference in its entirety herein and for all purposes.

### FIELD

The present disclosure relates generally to spinal implant assemblies utilizing universal shank heads having a common capture portion geometry, and that are configured for connection with an array or collection of pivoting and non-pivoting but axially rotatable (i.e., monoaxial) receiver sub-assemblies having different functionalities, and their use in surgery involving vertebral body stabilizations with spinal fixation systems.

### BACKGROUND

Spinal implants in general, and bone anchors or screws in particular, are used in many types of spinal surgery in order to secure various implants to vertebrae along the spinal column for the purposes of treating spinal disorders, such as degenerative conditions and deformities, and also for stabilizing and/or adjusting spinal alignment. A common mechanism for providing vertebral support is to implant the bone screws into certain bones which then, in turn, support a longitudinal structure such as an elongate rod, or are supported by such a rod. Although both closed-ended and open-ended spinal implants, such as bone screws and hooks, are known, the open-ended spinal implants can be particularly well suited for connections to rods and connector arms because such rods or arms do not need to be passed through a closed bore, but rather can be laid or urged into an open channel within the head or receiver of such a screw, hook, or connector. For example, open-ended bone screws generally comprise an anchor portion, such as a threaded shank, connected to a head or receiver having a pair of upwardly-projecting branches or arms which form a yoke that defines a slot or channel configured to receive the rod. The slot or channel could have different shapes, such as, a U-shape or a square shape. Moreover, the threaded shanks of the bone screws can also be replaced with hooks or other types of bone anchors or connectors to form a variety of different types of spinal implants, also having open ends for receiving rods or portions of other structures, and wherein such implants can facilitate surgical techniques performed with different spinal fixation systems.

Early bone screws used in spinal surgery generally had a yoke-shaped ‘head’ that was integrally formed or “fixed” with the threaded shank, and therefore immovable. Because the fixed head could not be moved relative to the shank, these fixed bone screws needed to be favorably positioned in the spine. Otherwise, the elongate rod would need to be bent in order for it to be placed within the rod-receiving channels of multiple implants due to their alignment. Given the highly curved shape of the spines of some patients, however, this is sometimes very difficult or impossible to do. Therefore, polyaxial (i.e., multiplanar), uni-planar (i.e., monoplanar), and/or translatable pivotal bone screws or bone anchor assemblies, were developed and are now commonly preferred. Open-ended polyaxial bone screw assemblies typically allow for pivoting and rotation of the connected but completely separate yoke-shaped receiver or receiver sub-assembly about an enlarged spherical ‘head ’ or upper capture portion of the threaded shank or bone anchor in one or more planes, until a desired rotational and pivotal position of the receiver is achieved relative to the shank. This can be accomplished by manipulating the position of the receiver relative to the shank during a final stage of a medical procedure when the elongate rod or other longitudinal connecting member is inserted into the receiver or receiver sub-assembly, followed by a locking set screw, a plug, a closure, or other type of locking mechanism known in the art.

It is understood that spinal fixation systems generally include a variety of components that require some assembly, such as the various types of bone anchors, the rods or connector arms, and the closures or plugs with the receivers or receiver sub-assemblies, with each component having specific features with respect to structure and function. Moreover, the receiver sub-assemblies can further include components in addition to the receiver itself, such as pressure inserts, spring rings, separate retainers, and other components of different types that are operable to connect these receiver sub-assemblies with the heads of the bone anchors. The pressure inserts, rings, retainers, and other components can be pre-assembled together within the receivers to form the receiver sub-assemblies that are ready for further assemblage with the bone anchors, and eventually with the rods or connector arms and the closures or plugs.

Some designs provide for the threaded shanks or other types of bone anchor to be bottom loaded into the receiver sub-assemblies. With bottom loaded bone anchor assemblies, for example, some designs known in the art require a separate retainer to hold the shank within the receiver, with the receiver having a bottom opening large enough to allow for the head or upper capture portion of the threaded shank or bone anchor to be uploaded into the central bore or cavity of the receiver. Other types of bottom loaded bone anchor assemblies do not include the separate retainer, however, and instead include a receiver having a lower portion with a bottom opening that is configured to directly threadably mate with the head or upper capture portion of the shank that can be configured as a threaded spherical head to provide for polyaxial or multiplanar motion.

Further to the above, bottom loaded bone anchor assemblies can also be fully assembled by the spinal company or distributor before being shipped to a hospital, so as to help with inventory management, or can be shipped as a modular array of multiple separate and different shanks and a fewer number of pre-assembled receiver sub-assemblies that can then be fully assembled, for example, at the hospital or surgical center during a surgery, thereby saving costs. Additionally, the modular spinal implants can be fully assembled at the hospital either before insertion into the patient, or after the threaded shank or bone anchor has been inserted into the patient, such as with robotic assistance or directly by a robot. The different techniques or approaches for the insertion and assembly of the modular parts of the bone anchor assemblies can be described as ex-vivo and in-vivo, respectively.

### SUMMARY

The present disclosure is generally directed to bone anchor assemblies that include a two-piece receiver comprising an upper body having an open channel for receiving a rod and an annular lower end with a downwardly-extending first connection structure, and a cylindrical base having a spherical seating surface adjacent a bottom opening and an upwardly-extending second connection structure configured for coupling with the first connection structure to secure the cylindrical base to the upper body. The assemblies also include bone anchors comprising a capture portion, such as a universal shank head, and an anchor portion opposite the capture portion configured for attachment to the bone of a patient. The assemblies further include retaining structures configured to secure the capture portion within the cylindrical base, and pressure inserts having an upper surface configured to receive the rod and a lower surface configured to engage an upper surface of the capture portion of the bone anchor. In each embodiment of the bone anchor assemblies the retaining structure is positionable into the cylindrical base and the pressure insert is positionable into the upper body prior to the cylindrical base and the upper body being coupled together to complete the two-piece receiver and receiver sub-assembly.

Additional embodiments of the present disclosure will be better understood upon review of the detailed description set forth below taken in conjunction with the accompanying drawing figures, which are briefly described as follows.

### BRIEF DESCRIPTION OF THE DRAWINGS

FIG. 1 is an exploded perspective view of a multiplanar embodiment of a bone anchor assembly, in accordance a representative embodiment of the present disclosure.

FIG. 2 is a perspective view of the bone anchor of the multiplanar bone anchor assembly of FIG. 1.

FIG. 3 is a close-up perspective view of the capture portion of the bone anchor of FIG. 2.

FIG. 4 is a cross-sectional view of the of the bone anchor of FIG. 2.

FIG. 5 is a close-up cross-sectional view of the capture portion of the bone anchor of FIG. 2.

FIG. 6 is a perspective view of the upper body of the two-piece receiver of the multiplanar bone anchor assembly of FIG. 1.

FIG. 7 is a top view of the upper body of FIG. 6.

FIG. 8 is a cross-sectional side view of the upper body of FIG. 6.

FIG. 9 is another cross-sectional side view of the upper body of FIG. 6.

FIG. 10 is a perspective view of the cylindrical base of the two-piece receiver of the multiplanar bone anchor assembly of FIG. 1.

FIG. 11 is a cross-sectional view of the cylindrical base of the two-piece receiver of FIG. 10.

FIG. 12 is an exploded cross-sectional view of the two-piece receiver of the multiplanar bone anchor assembly of FIG. 1 prior to the assembly of the cylindrical base to the upper body.

FIG. 13 is a cross-sectional view of the assembled two-piece receiver of FIG. 12 after the assembly of the cylindrical base to the upper body.

FIG. 14 is a perspective view of the ring retainer of the multiplanar bone anchor assembly of FIG. 1.

FIG. 15 is a cross-sectional view of the ring retainer of FIG. 14.

FIG. 16 is a perspective view of the capture ring of the multiplanar bone anchor assembly of FIG. 1.

FIG. 17 is a partially-sectioned perspective view of the ring retainer of FIG. 14 and the capture ring of FIG. 15 after assembly together into a multiplanar retainer sub-assembly.

FIG. 18 is a perspective view of the pressure insert of the multiplanar bone anchor assembly of FIG. 1.

FIG. 19 is a bottom perspective view of the pressure insert of FIG. 18.

FIG. 20 is an exploded partially-sectioned perspective view of the components of a multiplanar receiver sub-assembly prior to their pre-assembly into a shipping state configuration.

FIG. 21 is a partially sectioned front perspective view of the components of the multiplanar receiver sub-assembly of FIG. 20, showing the multiplanar retainer sub-assembly being seated within the cylindrical base and the pressure insert being uploaded into the upper body of the two-piece receiver.

FIG. 22 is a cross-sectional front view of the pre-assembled upper body and insert of FIG. 21.

FIG. 23 is a cross-sectional front view of the pre-assembled cylindrical base and multiplanar retainer sub-assembly of FIG. 21.

FIG. 24 is a cross-sectional front view of the components of the multiplanar receiver sub-assembly of FIG. 20 after the upper body and cylindrical base of the two-piece receiver have been connected together.

FIG. 25 is a cross-sectional front view of the components of the multiplanar receiver sub-assembly of FIG. 25 after the pressure insert has been downwardly deployed into engagement with the multiplanar retainer sub-assembly to form the pre-assembled multiplanar receiver sub-assembly in the shipping state configuration.

FIG. 26 is a partially sectioned front view of the multiplanar receiver sub-assembly of FIG. 25 positioned above the universal capture portion of a bone anchor.

FIG. 27 is a partially sectioned front view of the multiplanar receiver sub-assembly moving downward onto the universal capture portion of the bone anchor (or the capture portion moving upward into the receiver sub-assembly), showing the universal capture portion of the bone anchor having been pushed upward through the seated multiplanar retainer sub-assembly to engage the bottom surface of the pressure insert, until the capture ring snaps into the retainer recess to capture the universal capture portion within the multiplanar receiver sub-assembly.

FIG. 28 is another partially sectioned front view of the multiplanar receiver sub-assembly of FIG. 27, showing the pressure insert being moved downward within central bore of the two-piece receiver until the lower surface of the pressure insert becomes engaged with the upper surface of the ring retainer of the seated multiplanar retainer sub-assembly.

FIG. 29 is a perspective view of the multiplanar bone anchor assembly, fully assembled with the elongate rod and closure and with the bone anchor in a non-articulated position relative to the receiver.

FIG. 30 is a cross-sectional side view of the fully-assembled multiplanar bone anchor assembly of FIG. 29, with the bone anchor in an articulated position relative to the receiver.

FIG. 31 is an exploded perspective view of a monoaxial (i.e., substantially non-pivotal but axially rotatable) embodiment of a bone anchor assembly, in accordance with another representative embodiment of the present disclosure.

FIG. 32 is a perspective view of the upper body of the two-piece receiver of the monoaxial bone anchor assembly of FIG. 31.

FIG. 33 is a bottom perspective view of the upper body of FIG. 32.

FIG. 34 is a cross-sectional side view of the upper body of FIG. 32.

FIG. 35 is another cross-sectional side view of the upper body of FIG. 32.

FIG. 36 is a perspective view of the cylindrical base of the two-piece receiver of the monaxial bone anchor assembly of FIG. 31.

FIG. 37 is a bottom perspective view of the cylindrical base of FIG. 36.

FIG. 38 is a cross-sectional view of the assembled two-piece receiver of the monoaxial bone anchor assembly of FIG. 31.

FIG. 39 is a perspective view of the pressure insert of the monoaxial bone anchor assembly of FIG. 31.

FIG. 40 is a bottom perspective view of the pressure insert of FIG. 39.

FIG. 41 is a side perspective view of the pressure insert of FIG. 39.

FIG. 42 is a cross-sectional side view of the pressure insert of FIG. 39.

FIG. 43 is a perspective view of the ring retainer of the monoaxial bone anchor assembly of FIG. 31.

FIG. 44 is a cross-sectional view of the ring retainer of FIG. 43.

FIG. 45 is a perspective view of the capture ring being assembled into the monoaxial ring retainer of FIG. 43.

FIG. 46 is a partially-sectioned perspective view of the ring retainer and the capture ring of FIG. 45 after assembly together into the monoaxial retainer sub-assembly.

FIG. 47 is an exploded partially-sectioned perspective view of the components of the monoaxial receiver sub-assembly prior to their pre-assembly into a shipping state configuration.

FIG. 48 is a close-up partially-sectioned perspective view of the components of the monaxial receiver sub-assembly of FIG. 47 showing the pressure insert being uploaded into the upper body of the two-piece receiver.

FIG. 49 is a partially-sectioned perspective view of the components of the monoaxial receiver sub-assembly of FIG. 47 after the pressure insert has been downwardly deployed into engagement with the multiplanar retainer sub-assembly to form the pre-assembled monoaxial receiver sub-assembly in the shipping state configuration.

FIG. 50 is a close-up partially-sectioned perspective view of the upper body and pressure insert of the monoaxial receiver sub-assembly of FIG. 49, showing pressure insert downwardly deployed within the central bore of the two-piece receiver.

FIG. 51 is a partially sectioned front view of the monoaxial receiver sub-assembly of FIG. 50 positioned above the universal capture portion of a bone anchor.

FIG. 52 is a partially sectioned front view of the monoaxial receiver sub-assembly moving downward onto the universal capture portion of the bone anchor (or the capture portion moving upward into the receiver sub-assembly), showing the universal capture portion of the bone anchor having been pushed upward through the seated monoaxial retainer sub-assembly to engage the bottom surface of the pressure insert, prior to the capture ring snaping into the retainer recess to capture the universal capture portion within the monoaxial receiver sub-assembly.

FIG. 53 is another partially sectioned front view of the monoaxial receiver sub-assembly of FIG. 52, showing the pressure insert being moved downward within central bore of the two-piece receiver until the lower surface of the pressure insert becomes engaged with the upper surface of the ring retainer of the seated monoaxial retainer sub-assembly.

FIG. 54 is a perspective view of the monoaxial bone anchor assembly, fully assembled with the elongate rod and closure and with the bone anchor in a non-articulated position relative to the receiver.

FIG. 55 is a cross-sectional front view of the fully-assembled monoaxial bone anchor assembly of FIG. 54.

Those skilled in the art will appreciate and understand that the various features and structures or components of the bone anchor assemblies shown in the drawings described above, together with their relative relationships, interconnections and functions, can be interpreted as being drawn to scale. Nevertheless, it is also understood that the representative embodiments of the present disclosure disclosed and claimed herein are not limited to the precise structures and interrelationships of the features and components shown in the drawing figures, and that the dimensions, relative positions, and interconnections between the illustrated features and components may also be expanded, reduced, re-shaped, or otherwise revised or altered as needed to more clearly illustrate the structure of the embodiments depicted therein or the functions of the various features and components, as described below. Again, it is foreseen that some parts and features are interchangeable in their arrangement between the different embodiments disclosed.

### DESCRIPTION OF THE INVENTION

The following description, in conjunction with the accompanying drawings, is provided as an enabling teaching of pivotal bone anchors assemblies having bone anchors with ‘universal’ shank heads configured to cooperate with separate retaining structures that, in turn, have been pre-loaded into two-piece receivers to form receiver sub-assemblies with different functionalities, and with the universal shank heads being bottom-loaded into the pre-assembled receiver sub-assemblies. As described below, the representative type of universal shank head or capture portion of a bone anchor illustrated herein includes a horizontal capture recess extending into and circumferentially around a mid-portion of the capture portion, and which is bounded above and below by outer slidable surfaces. Nevertheless, it is foreseen that other suitable capture portion geometries can be used with modified versions of the retaining structures, two-piece receivers and bottom-loaded receiver sub-assemblies described herein, and which alternative capture portion geometries and receiver sub-assemblies are considered to fall within the scope of the present disclosure.

The bone anchors are generally configured for use with a collection or array of complementary pivotal and non-pivotal receiver sub-assemblies in a spinal fixation system. In particular, the collection can include different types of receiver sub-assemblies that can be coupled to the universal shank heads of the bone anchors to form bone anchor assemblies having different and specialized modes of movement, degrees of freedom, or modalities (with the terms ‘mode’, ‘modality’, and ‘multi-modal’, etc., being used herein to describe the way in which something moves), including but not limited to pivoting and non-pivoting but axially rotatable (e.g. monoaxial) movement of the receiver sub-assembly relative to a shank or bone anchor that is further configured for implantation into the bone of a patient. The description also includes one or more methods for assembling and employing the bone anchors with the multi-modal collection of receiver sub-assemblies. As described below, individual bone anchor assemblies, systems, and/or methods of assembly and/or use of the present disclosure for this representative type of universal shank head can provide significant advantages and benefits over other pivotal and/or non-pivotal bone anchors and spinal fixation systems known in the art due to, in one aspect, the degree of versatility and adaptability provided by the shank head universality (i.e. all of the shank heads having a common geometry that is connectable with each type of receiver sub-assembly that has its own predetermined combination of degrees of freedom and operational functionalities). The recited advantages are not meant to be limiting in any way, however, as one skilled in the art will appreciate that other advantages and benefits may also be realized upon practicing the present disclosure.

Furthermore, those skilled in the relevant art will recognize that changes can be made to the disclosed embodiments for shank head universality, beyond those described, while still obtaining the beneficial results. It will also be understood and appreciated that some of the advantages and benefits of the described embodiment for the invention can be obtained by selecting some of the features (e.g., the structures or components) of the disclosed receiver sub-assemblies without utilizing other features, and that features from one sub-assembly embodiment may be interchanged or combined with features from other sub-assemblies in any appropriate combination. For example, any individual feature or collective features of method embodiments may be applied to apparatus, product or system embodiments, and vice versa. Likewise, structural elements or functional features from one embodiment may also be combined with or replaced by structural elements or functional features from one or more additional embodiments in any suitable manner. Those who work in the art will therefore recognize that many modifications and adaptations to the representative embodiments described herein are possible and may even be desirable in certain circumstances, and are to be considered part of the present disclosure. Thus, it will be appreciated that the present disclosure is provided as an illustration of the principles for the representative pivotal and non-pivotal bone anchor assemblies incorporating the universal shank head that are shown and discussed therein, since the scope of each invention disclosed herein is to be defined by their respective claims.

### Multiplanar Bone Anchor Assembly

Referring now in more detail to the drawing figures, wherein like parts are identified with like reference numerals throughout the several views, FIG. 1 is an exploded perspective view of one representative embodiment of a multiplanar pivotal bone anchor assembly 10 that includes a bone anchor 50, such as a threaded shank, having a universal shank head or capture portion 60, and an anchor portion 84 opposite the capture portion 60 configured for securement within or attachment to the bone of a patient. The universal capture portion 60 of the illustrated embodiment further includes a horizontal capture recess 70 extending into and circumferentially around a mid-portion therefore, and which capture recess 70 is bounded above and below by upper and lower slidable surfaces 68, 74, respectively, that can be lateral facing frusto-conical outer surfaces devoid of any parallel planar surfaces.

The multiplanar bone anchor assembly 10 also includes a multiplanar two-piece receiver 100 or housing having a cylindrical base 140 defining the lower portion of an internal cavity 135 that is configured to receive the capture portion 60 of the bone anchor 50, and an upper body 120 that includes a pair of upright arms 110 integrally formed with and extending upward from an annular lower end 125 to define an open rod channel 105 configured to receive an elongate rod 4 (not shown). The annular lower end 125 can further define the upper portion of the internal cavity 135, and the annular lower end 125 and upright arms 110 can together define a central bore 115 that extends upward from the internal cavity 135 through the rod channel 105 to the tops of the upright arms 110, and which is centered about the vertical centerline axis 101 of the multiplanar receiver.

The two-piece receiver 100 can be pivotably secured to the capture portion 60 of the bone anchor 50 with a number of separate internal components that have been pre-assembled into the internal cavity 135 and the central bore 115 to form a multiplanar receiver sub-assembly 12. These internal components can include a ring retainer 150 with a separate open capture ring 170 secured therein (which ring retainer 150 and capture ring 170 together define a pivoting or articulating multiplanar retainer sub-assembly 14) and a pressure insert 180. After the elongate rod has been positioned within the lower portion of the rod channel 105, a closure 90 can be threadably or otherwise secured into an upper portion of the rod channel 105/central bore 115 to apply pressure to an upper surface of the elongate rod, such as by direct contact therebetween, thereby locking the elongate rod and the multiplanar bone anchor assembly 10 together into a final locked position.

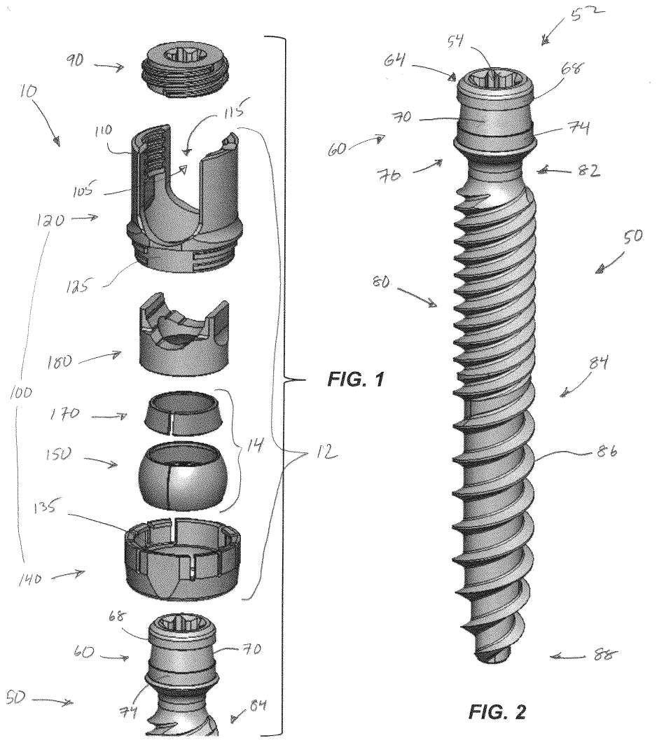

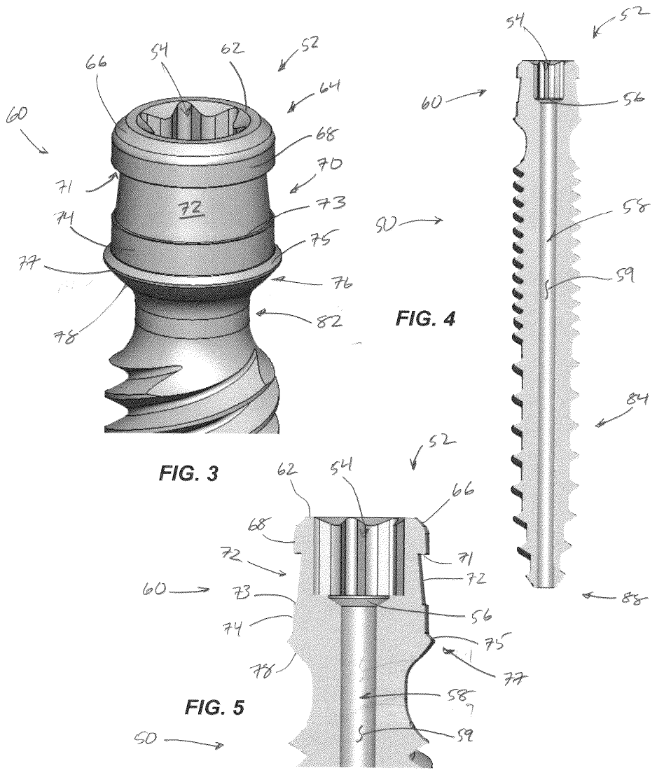

As shown in the exploded perspective view of FIG. 1 and isolated views of FIGS. 2-5, the bone anchor 50 has a capture portion 60, or type of universal shank head, at a proximal end 52, and a body 80 extending distally from the capture portion 60 with an attachment or anchor portion 84 at a distal end 88 configured for fixation to the bone of a patient. In one aspect the body 80 of the bone anchor 50 can comprise both the anchor portion 84 as well as a narrow neck portion 82 extending longitudinally between the anchor portion 84 and the capture portion 60. Although shown in the figures as a shank body 80 with bone engagement threads 86, it is foreseen that the anchor portion 84 of the shank body 80 could also be configured as a hook blade, or another type of bone attachment structure, extending downward from the neck portion 82. In one aspect the universal capture portion 60 or shank head can be common different types of bone anchors included a spinal fixation system as described above, regardless of the size or type of the anchor portion extending downward or below the capture portion 60.

At its upper end, the universal capture portion 60 or shank head includes an annular horizontally-planar top surface 62 that surrounds an internal drive feature or drive socket 54. Extending downwardly and outwardly from the top surface 62 is an upper curvate section 64 that is radiused, chamfered or beveled with respect to the horizontal flat top surface 62, and which, in one aspect, can ease the entry of the proximal end 52 of the bone anchor 50 into the center aperture of the capture ring 170 positioned within the ring retainer 150 (see FIG. 1). Below the upper curvate section 64 are an upper outer slidable surface 68 and a lower outer slidable surface 74 centered on the bone anchor's longitudinal or spin axis 51, and which are separated by a horizontal capture recess 70 extending into and circumferentially around a mid-portion of the capture portion 60. The capture portion 60 can further include a lower curvate section 76 that curves downwardly and inwardly from the lower outer slidable surface 74 toward the narrow neck portion 82 of the body 80 of the bone anchor. In one aspect the lower curvate section 76 can extend radially outward beyond the lower end portion of the lower outer slidable surface 74 to create an outer lip structure 77 that can be further defined, in part, by an upwardly-and outwardly-facing beveled lip surface 75. As described above, these outer surfaces of the capture portion 60 are generally devoid of parallel flat or planar outer side surfaces or sections that might require a type of keyed entry through the bottom opening of the receiver.

The upper curvate section 64 and the lower curvate section 76 of the capture portion 60 of the bone anchor 50 can further include upper and lower spherical surfaces 66, 78 that together define upper and lower spherical extensions, respectively, of a spherical outer surface of the ring retainer 150, so as to form a substantially spherical capture head when the ring retainer 150 is coupled to the capture portion 60. As described in detail below, the upper spherical section 64 can be engaged by the complementary-shaped bottom surface of the pressure insert 180 upon assembly of the bone anchor 50 to the multiplanar receiver sub-assembly 12, and the lower spherical section 76 can engage the complementary-shaped spherical seating surface of the multiplanar receiver 100 upon articulation of the bone anchor 50 relative to the multiplanar receiver sub-assembly 12.

The upper and lower slidable surfaces 68, 74 can be straight cylindrical surfaces or preferably frusto-conical surfaces having a slight taper angle devoid of any flat surfaces, and can have different lengths as measured along the longitudinal axis of the bone anchor 50. When formed as frusto-conical surfaces (as shown), the outer slidable surfaces 68, 74 can be widest toward the lower end of the capture portion 60 (i.e. the end that is closest to the neck 82 or anchor portion 84 of the bone anchor 50), and then can narrow or taper while moving upward toward the upper end 52 and top surface 62 of the capture portion 60. It will be appreciated that the tapered slidable surfaces 68, 74 may also facilitate the insertion of the capture portion 60 into the center aperture of the capture ring 170, so as to be easier than the insertion of a capture portion having a straight cylinder shape into a center aperture that can, in some aspects, also be defined by non-tapered inner cylindrical surfaces. Although the upper slidable surface 68 and the lower slidable surface 74 are shown as having a common straight taper angle extending between the two surfaces of about one degree, it is foreseen that in other embodiments the upper slidable surface 68 and the lower slidable surface 74 can each define their own tapered surface having a taper angle ranging between about 0.5 degrees to about five degrees, or greater. Moreover, each of the upper and lower slidable surfaces 68, 74 may have a taper angle that is same or different from the other, and may also have an outer diameter at a lower end that is the same or different from the other.

As shown in the drawings, the upper and lower slidable surfaces 68, 74 can bracket the horizontal capture recess 70 extending into and circumferentially around the mid-portion of the capture portion 60, with the capture recess 70 having a height between an upper step surface 71 and a lower step surface 73 that can be significantly greater than its depth. For example, the height of the capture recess can be three to four times greater than its depth. In addition, the outwardly-facing inner recessed surface 72 of the capture recess 70 that extends between the upper step surface 71 and the lower step surface 73 can be tapered, with a taper angle ranging between about three degrees and about nine degrees, thereby resulting in the upper step surface 71 having a greater depth or overhang than that of the lower step surface 73. Although shown with an inner recessed surface 72 that is tapered, it is foreseen that the capture recess can have an inner recessed surface with a different profile, including but not limited to a straight cylindrical profile, a curved, curvate or curvilinear profile, a spherical profile, and the like. In one aspect the upper step surface 71 can also have an outwardly-and downwardly-beveled profile to form an undercut or a dove-tail like engagement with a top surface of the capture ring 170 that serves to keep the capture ring 170 in position upon connection with the capture recess 70 of the capture portion 60, as described below.

As described above, the top surface 62 of the capture portion 60 can be an annular planar top surface that surrounds an internal drive feature 54 or drive socket. The illustrated internal drive feature 54 is an aperture formed in the top surface 62, and in one aspect can be a multi-lobular or star-shaped aperture, such as those sold under the trademark TORX, or the like, having internal faces 55 designed to receive a multi-lobular or star-shaped tool for rotating and driving the bone anchor 50 into the vertebra. It is foreseen that such an internal tool engagement structure or drive feature 54 may take a variety of tool-engaging forms and may include one or more apertures of various shapes, such as a pair of spaced apart apertures or a hex shape designed to receive a hex tool (not shown) of an Allen wrench type. The seat or base surface 56 of the drive feature 54 can be disposed perpendicular to the shank axis, with the drive feature otherwise being coaxial with the longitudinal axis 52 of the bone anchor 50. In operation, a driving tool is received in the internal drive feature 54, being seated at the base surface 56 and engaging the internal faces 55 of the drive feature for rotating and driving the anchor portion 84 of the bone anchor into the vertebra, either before or after the bone anchor 50 is attached or coupled to the multiplanar receiver sub-assembly 12. If attached, the threaded anchor portion 84 of the body 80 of the bone anchor can be driven into the vertebra with the driving tool extending downward through both the central bore 115 of the multiplanar receiver 100 and the central aperture of the pressure insert 180 of the multiplanar receiver sub-assembly 12.

In one aspect the bone anchor 50 or shank can be cannulated (and also fenestrated for the application of bone cement, or even expandable) with a narrow axial bore 58 or aperture extending through the entire length thereof and centered about the longitudinal axis 51 of the shank. The axial bore 58 can be defined by an inner cylindrical sidewall 59 with a lower circular opening 89 at the distal end 88 of the shank body 80, and an upper circular opening 56 communicating with the internal drive socket 54 at the internal base surface thereof, and is coaxial with the body 80 and the capture portion 60 of the bone anchor 50. The axial bore 58 provides a passage through the shank interior for a length of wire (not shown) inserted into the vertebra prior to the implantation of the anchor portion 84 of the bone anchor 50, the wire providing a guide for insertion of the anchor portion 84 into the vertebra. The axial bore 58 can also provide for a pin to extend therethrough and beyond the distal end 88 of the shank body 80, the pin being associated with a tool to facilitate insertion of the body 80 of the bone anchor into the vertebra.

To provide a biologically active interface with the bone, the body 80 of the bone anchor 50, including both the threaded anchor portion 84 and the neck 82, may be coated, perforated, made porous or otherwise treated or textured. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca3(PO4)2, tetra-calcium phosphate (Ca4P2O9), amorphous calcium phosphate and hydroxyapatite (Ca10(PO9)6(OH)2). Coating with hydroxyapatite, for example, is desirable as hydroxyapatite is chemically similar to bone with respect to mineral content and has been identified as being bioactive and thus not only supportive of bone ingrowth, but actively taking part in bone bonding.

Additional detailed discussion of the structures and features of the bone anchor 50 and its universal capture portion 60, as well as its assembly into and interactions with the structures and features of the pivoting or articulating retainer sub-assembly 14 that includes the ring retainer 150 having the separate open capture ring 170 secured therein, is provided in co-owned U.S. Pat. No. 12,053,209, filed Jan. 18, 2023, which is incorporated by reference in its entirety herein and for all purposes.

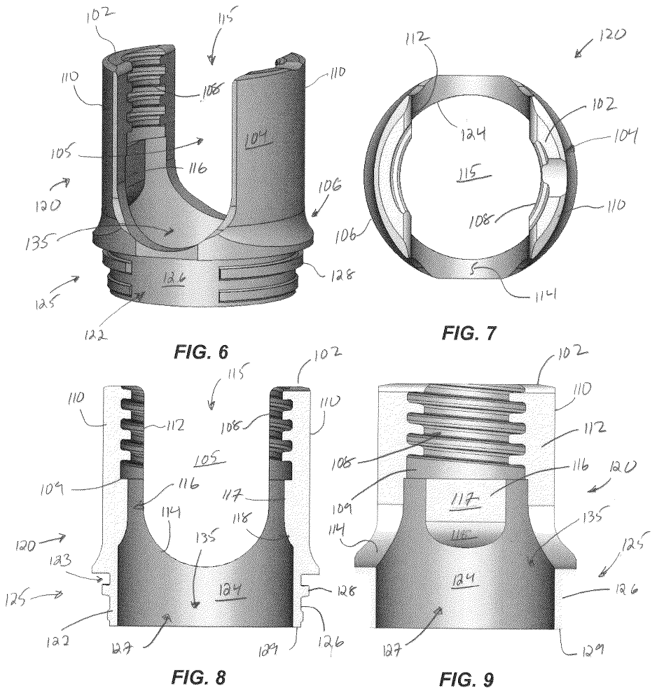

With reference now to FIGS. 6-13, the receiver 100 can be formed as a two-piece body comprising the cylindrical base 140 that can be attached or coupled to the upper body 120 after the retainer sub-assembly 14 has been pre-assembled into the cylindrical base 140 and the pressure insert 180 had been pre-assembled into the upper body 120. It will be appreciated that both the upper body 120 and the cylindrical base 140 can have substantially-cylindrical shapes that can also include flat portions, grooves or recesses, flared portions, flanges, and apertures, etc., formed into outer surfaces, as well as an annular sub-structure (or first connection structure) extending downward at the lower end portion of the upper body and an annular super-structure (or second connection structure) extending upward from an upper end portion of the cylindrical base 140.

Shown in FIGS. 6-9 is the upper body 120 of the two-piece receiver 100 that includes an upper portion of the internal cavity 135, as defined by the annular lower end 125, that receives the lower portion of the pressure insert 180. The upper body 120 also includes the open rod channel 105, as defined by the pair of upright arms 110, and the central bore 115 that extends upwardly from the internal cavity 135 through the rod channel 105 to the top surfaces 102 of the upright arms 110. As described in more detail below, the upper portion of the internal cavity 135 and internal surfaces of the central bore 115 of the upper body 120 can include features that are complementary with the outer surfaces of the pressure insert 180, so as to allow the pressure insert 180 to be slidably received and/or secured therein and to be maintained in alignment with respect to the open rod channel 105.

For example, the annular lower end 125 of the upper body 120 can include a downwardly-extending annular skirt 122 with a circular lower edge surface 129 that defines a lower opening 127, and an inner cylindrical surface 124 that defines the lower portion of the central bore 115. The inner cylindrical surface 124 can extend upwardly into the open rod channel 105 until it reaches the runout groove 109 of a discontinuous guide and advancement structure 108 that is formed into upper portions of the internal surfaces of the upright arms 110. In one aspect the inner flange surfaces of the discontinuous guide and advancement structure 108 can define the narrowest portion of the central bore 115, while vertical planar end surfaces 112 on either side of the guide and advancement structure 108 and saddle surfaces 114 can define the front and back ends of the rod channel 105 that are adjacent the front and back faces of the receiver 100. As shown in the drawings, an inwardly-extending protrusion 116 with an upper planar alignment surface 117 and lower curvate transition surface 118 can also be formed into the internal surfaces of the upright arms 110 below the discontinuous guide and advancement structure 108.

The upright arms 100 of the upper body 120 of the two-piece receiver can further include outer side surfaces 104 that extend downwardly from the tops of the upright arms 100 to lower, outwardly-flared portions 106 located proximate the U-shaped saddle surfaces 114 that define the lower portion of the rod channel 105. Below the outwardly-flared portions 106 and U-shaped saddle surface 114, the annular skirt 122 of the annular lower end 125 can further include a lower outer cylindrical surface 126 with a plurality of arcuate ribs 128 extending outwardly therefrom. In one aspect the arcuate ribs 128 can comprises opposite sets of one or more arcuate ribs 128 extending outwardly below the upright arms 110, with unobstructed portions of the lower outer cylindrical surface 126 being located below the front and back openings of the rod channel 105.

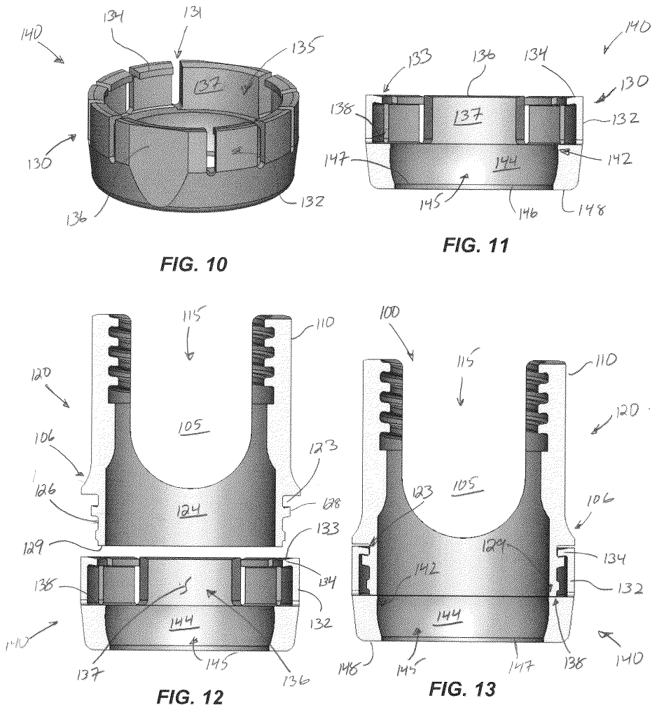

As shown in FIGS. 10-11, the cylindrical base 140 can define the lower portion of the internal cavity 135 that communicates with the bottom surface 148 of the cylindrical base 140 through a bottom opening 145. The lower portion of the internal cavity 135 can include a spherical seating surface 144 that is sized and shaped to closely receive the spherical outer surface of the ring retainer 150 of the retainer sub-assembly 14, as describe in more detail below. The seating surface 144 can include an overtravel lip portion 142 that is configured to extend above a hemisphere plane of the spherical outer surface of the ring retainer 150 to an internal ledge 138 that defines the upper border of the seating surface 144. In one aspect a tapered lower surface 146 can extend downwardly and outwardly, from the lower edge 147 of the spherical seating surface 144 to the bottom surface 148 of the cylindrical base 140, to define the bottom opening 145.

The cylindrical base 140 can also include an annular upper structure 130 configured to engage with the annular skirt 122 of the upper body 120 to secure the upper body 120 to the cylindrical base 140. The annular upper structure 130 can include opposing sets of upwardly-extending flexible curvate panels or spring tabs 132, separated by slots 131, and having inwardly-extending flanges or hook structures 134 at their upper ends configured to hook onto the upper surfaces of the arcuate ribs 128 extending outwardly from the lower outer cylindrical surface 126 of the upper body 120. In one aspect the opposing sets of spring tabs 132 can be separated by opposing front/back curvate panels 136 having a greater circumferential length around the annular upper structure 130, as well as an increased thickness in the radial direction, that can make the front/back curvate panels 136 more rigid, or provide them with greater stiffness, than the spring tabs 132. The front/back curvate panels 136 can further define interior partial cylindrical surfaces 137 configured to closely receive the unobstructed portions of the lower outer cylindrical surfaces 126 of the upper body 120 located below the front and back openings of the rod channel 105.

With reference to FIGS. 12-13, in one aspect the means or mechanism for coupling together the upper body 120 and the cylindrical base 140 can comprise a type of spring latch connection. For example, the inwardly-projecting hook structures 134 at the upper ends of the spring tabs 132 can have beveled upper edges 133 configured to first engage the underside surfaces of the arcuate ribs 128 (which can also be beveled surfaces) as the upper body 120 and cylindrical base 140 are moved into connection with each other. This can cause the spring tabs 132 to flex outwardly until the hook structures 134 can move upwardly across the arcuate ribs 128 and thereafter snap into the arcuate gaps or recesses 123 located between the upper surfaces of the arcuate ribs 128 and lower surfaces of the outwardly-flared portions 106 of the upper body 120, as shown in FIG. 13. At the same time the circular lower edge surface 129 of the upper body 120 can abut the internal ledge 138 of the cylindrical base 140, just above the overtravel lip portion 142, to complete the formation of the internal cavity 135. The un-obstructed portions of the lower outer cylindrical surface 126 located below the front and back openings of the rod channel 105 can also be closely received by the interior partial cylindrical surfaces 137 of the front/back curvate panels 136, so as to provide stiffness and strength to the assembled two-piece receiver 100 and to prevent rotation between the upper body 120 and the cylindrical base 140.

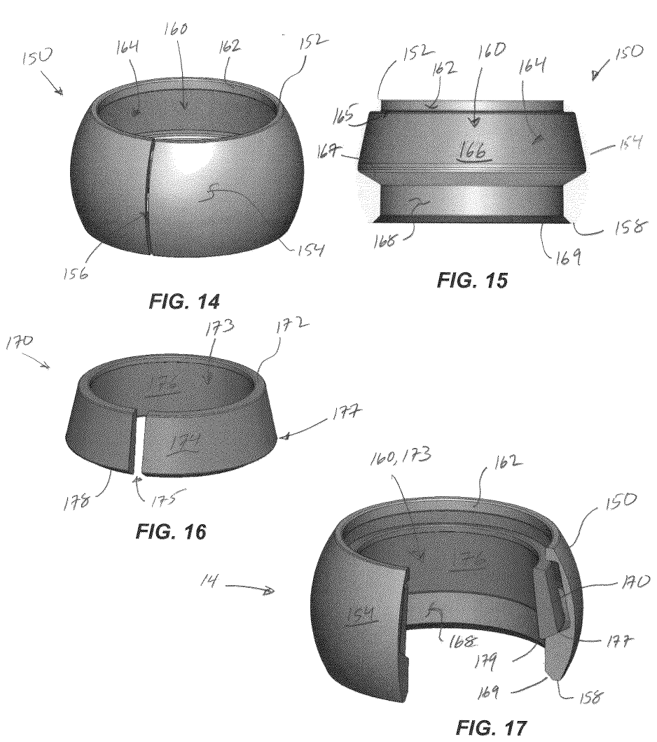

Illustrated in FIGS. 1, 14-15 and 17 is an example of a multiplanar embodiment for the ring retainer 150 comprising an O-ring body having a curvate outer surface, such as spherical outer surface 154 extending between a top annular or edge surface 152 and a bottom annular or edge surface 158, and a center aperture 160. The center aperture 160 can be defined by inner slidable surfaces 162, 168 that are configured to loosely slidably engage with the outer slidable surfaces 68, 74 of the capture portion 60 of the bone anchor 50 when the capture portion is fully uploaded into the ring retainer 150. The ring retainer 150 can also include an internal recess 164 that extends into and circumferentially around a mid-portion of the center aperture 160, and which can be defined by an upper annular surface 165, a recess sidewall surface 166, and a lower tapered or beveled surface 167. The center aperture 160 can further include a lower beveled surface 169 extending between the lower inner slidable surface 168 and the bottom annular or edge surface 158, and which can be complementary with the beveled lip surface 75 of the capture portion 60 of the shank 50. The ring retainer 150 can also include a slit or slot 156 extending through the thickness of the O-ring body, from the center aperture 160 through to the outer surface 154, to form an open ring retainer that is expandable so as to allow the capture ring 170 to be preloaded or positioned within the internal recess 164, and which is also compressible so as to allow the ring retainer 150 to be pressed into the spherical seating surface 144 of the cavity 135 of the multiplanar two-piece receiver 100 so as to provide for a pre-lock friction fit and alignment stability for the retainer sub-assembly 14 with respect to the two-piece receiver 100.

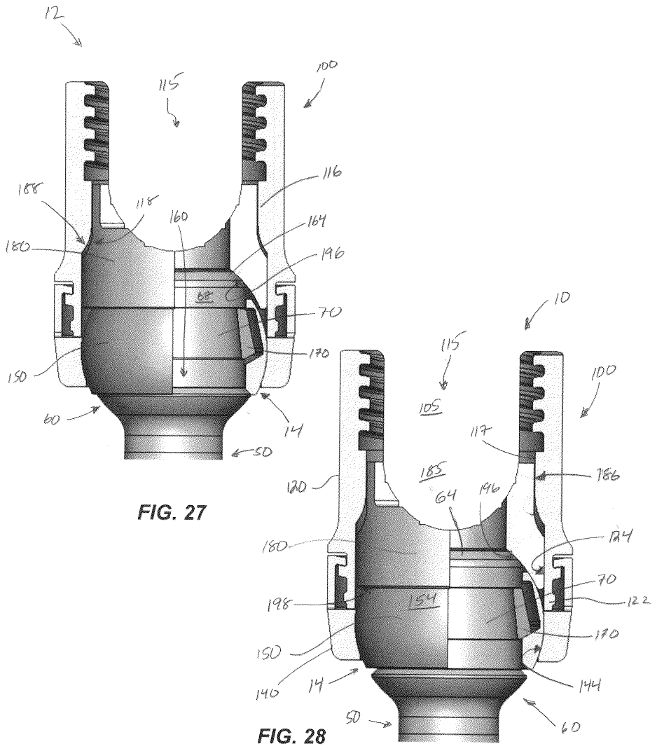

As described in more detail below, the ring retainer 150 is securable within the cavity 135 of the cylindrical base 140 with the spherical outer surface 154 being frictionally engaged with the spherical seating surface 144, and with the center aperture 160 being centered above the bottom opening 145 of the two-piece receiver 100. As noted above, the spherical outer surface 154 of the ring retainer 150 can also have the same radius as the lower spherical surface 78 of the lower curvate section 76 and the upper spherical surfaces 66 of the upper curvate section 64 of the capture portion 60 of the bone anchor 50, in which case the three spherical surfaces 154, 66, 78 may align with each other to form a substantially spherical capture head when the bone anchor 50 is coupled to the ring retainer 150. Moreover, the lower beveled surface 169 of the ring retainer 150 can be adjacent to, or even contacting, the beveled lip surface 75 of the capture portion 60 (described above) when the bone anchor 50 is coupled to the ring retainer 150 (as shown in FIGS. 27-28).

With the reference to FIGS. 2 and 16, the multiplanar pivotal bone anchor assembly 10 further includes a capture ring 170 comprising an open ring body having a slit or slot 175 that allows for expansion of the capture ring 170 during assembly of the bone anchor 50 to the multiplanar receiver sub-assembly 12. As illustrated in the drawing figures, the open ring body can have a variable height between an outwardly-and downwardly-beveled top surface 172 and an outwardly-and upwardly-beveled bottom surface 178. The open ring body can also have a variable width between a tapered outer surface 176 and a tapered inner surface 176 that is greater toward the bottom end of the capture ring 170, so as to define a non-rectangular wedge-shaped body in cross section. Moreover, the height of the capture ring 170, as measured along a central axis of the capture ring, can be at least twice its maximum width, as measured between the tapered inner surface 176 of the capture ring aperture 173 and a lower outer edge 177 that defines the junction between the tapered outer surface 174 and the beveled bottom surface 178. Indeed, in some aspects the height of the capture ring 170 can be three to four times the maximum width, so as to better control and direct the forces that are transferred downward from the upper portion of the capture portion of the shank to the lower portion of the ring retainer 150 and seating surface 144 of the multiplanar two-piece receiver 100.

Additional details regarding the structures and features of the ring retainer 150 and the capture ring 170 and their pre-assembly together into a pivoting or articulating retainer sub-assembly 14, as well as their subsequent interactions with the structures and features of the capture portion 60 of the bone anchor 50, the two-piece receiver 100, and the pressure insert 180, can be found in co-owned U.S. Pat. No. 12,053,209 described and incorporated by reference above.

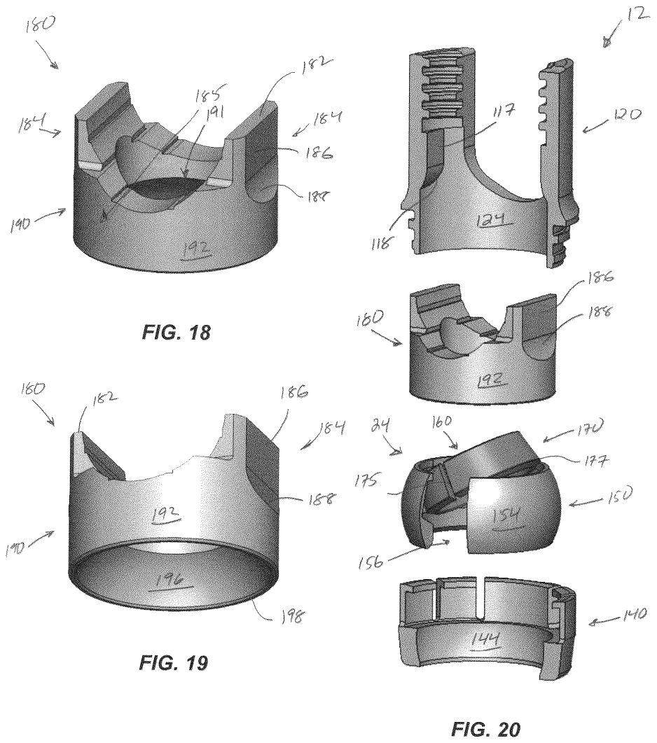

With the reference to FIGS. 2 and 18-19, the pressure insert 180 of the multiplanar pivotal bone anchor assembly 10 can comprise a generally-cylindrical insert body 190 with a center aperture 191 that is alignable with the vertical centerline axis of the two-piece receiver 100 and having a linear upper groove or insert channel 185 formed therein that is configured for engagement by the lower surface portion of the elongate rod. The upward-facing insert channel 185 can extend transversely across the top of the insert body 190 and the center aperture 191 to define two upwardly-extending side portions or insert arms 184 having top surfaces 182 located below a top surface of the elongate rod when the elongate rod is positioned with the receiver and insert channels 105, 185, respectively. In one aspect the two upwardly-extending side portions or insert arms 184 can further include opposite flat or planar outer surfaces 186 extending downward from the top surfaces 182 to outer curvate transition surfaces 188. Upon assembly together, the opposite planar outer surfaces 186 can be configured to slidably engage with the upper planar alignment surfaces 117 formed into the internal surfaces of the upright arms 110 of the upper body 120 of the two-piece receiver, so as to establish and maintain the insert channel 185 in alignment with the open rod channel 105 of the upper body 120. In addition, the outer curvate transition surfaces 188 of the pressure insert 180 can be configured to abut the complimentary inner curvate transition surfaces 118 formed into the internal surfaces of the upright arms 110 of the upper body 120 when the pressure insert is in its most upwardly-advanced position (FIGS. 22 and 27).

The insert body 190 of the pressure insert 180 can also include a lower cylindrical outer surface 192 extending downward to an annular bottom edge surface 198, and which is configured for slidable engagement within the upper inner cylindrical surface 124 of the upper body 120 of the two-piece receiver 100. As shown in the drawings, the insert body 190 can further include a downward-facing concave lower surface 196 that is configured to engage the spherical or curvate upper portion 64 of the capture portion 60 upon the capture portion's uploading through the bottom opening 145 of the two-piece receiver 100.

Illustrated in FIG. 20 are the individual components of the multiplanar pivotal bone anchor assembly 10 that, in many embodiments, can be pre-assembled together into a receiver sub-assembly 12 at a factory or manufacturing facility, prior to shipping to a spine company or a hospital or surgery center and engagement with the capture portion of the bone anchor in the surgical setting. As described above, these components generally include the multiplanar two-piece receiver 100 comprising the cylindrical base 140 and upper body 120, the retainer sub-assembly 14 that incorporates the ring retainer 150 with the separate open capture ring 170 secured therein, and the pressure insert 180. In one aspect the two-piece receiver 100, the retainer sub-assembly 14, and the insert 180 being pre-assembled into a multiplanar receiver sub-assembly 12 can be further defined as the shipping state configuration for the ‘modular’ bone anchor assembly, as described herein and commonly understood in the art. It will be appreciated, however, that in other embodiments the shipping state configuration can include the additional assembly of the multiplanar receiver sub-assembly together with the bone anchor at the factory or manufacturing facility or the spine company. It will also be appreciated that in yet other embodiments the individual components described above can also be pre-assembled into the receiver sub-assembly at the hospital or surgery center prior to implantation in a patient.

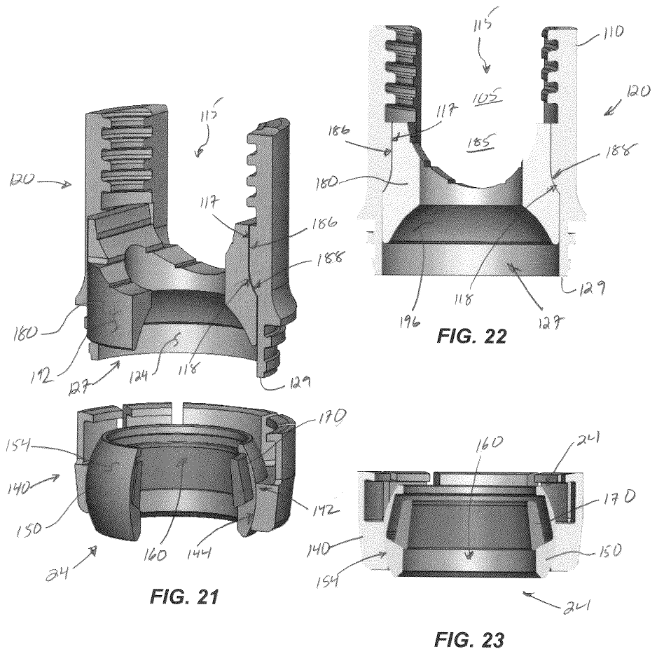

To begin the pre-assembly of the receiver sub-assembly 12, the capture ring 170 can first be installed or positioned into the internal recess 164 of the ring retainer 150 to form the retainer sub-assembly 14, as shown in FIG. 20. In particular, the capture ring 170 can be placed above the ring retainer 150 in preparation for top loading into the center aperture 160 of the ring retainer 152 (although bottom loading into the ring retainer is also contemplated). The ring retainer 150 can then be expanded and the slot 156 opened until the diameter of the center aperture 160 is greater than the outer diameter of the lower outer edge 177 of the capture ring 170. In one aspect the capture ring 170 can also be slightly compressed so that the width of the capture ring slot 175 and the outer diameter of the lower outer edge 177 is less than what it would be in its neutral or free-standing state. As shown the drawings, the capture ring 170 may then be tilted and inserted into the center aperture 160 of the ring retainer 150 at an angled orientation. The ring retainer 150 can then be released to close back toward its neutral state to capture the now-horizontal capture ring 170 within the internal recess 164, as shown in FIGS. 21 and 23.

It will be understood that the forced expansion of the ring retainer 150 shown in FIG. 20 can result in a marginal inelastic deformation of the O-ring body of the ring retainer 150, so that the diameter of the spherical outer surface 154 in the free and neutral state is now greater than what is was before the expansion. As described below, this deformation can be advantageously employed in subsequent assembly steps to establish a compressive friction engagement between the spherical outer surface 154 of the ring retainer 150 and the spherical seating surface 144 of the cylindrical base 140 of the two-piece receiver 100.

With continued reference to FIGS. 20-23, the pressure insert 180 can be uploaded into the central bore 115 of the upper body 120 of the two-piece receiver 100 through the lower opening 127 defined by the circular lower edge 129, with the lower cylindrical outer surface 192 of the pressure insert entering into slidable engagement with the inner cylindrical surface 124 of the upper body 120 and the insert channel 185 being generally aligned with the rod channel 105. The pressure insert 180 can then be moved upwardly within the central bore 115 until the opposite planar outer surfaces 186 on the arms 184 of the pressure insert 180 slidably engage with the upper planar alignment surfaces 117 on the protrusion 116 that extends inwardly into the central bore 115, so as to establish and maintain the insert channel 185 in alignment with the open rod channel 105 of the upper body 120. With continued upward movement of the pressure insert 180, the outer curvate transition surfaces 188 of the pressure insert 180 can eventually abut the complimentary inner curvate transition surfaces 118 of the protrusion 116, as shown in FIGS. 21 and 22, so as to provide a stop surface that restricts further upward movement of the pressure insert 180 relative to the upper body 120. In one aspect the opposite planar outer surfaces 186 on the arms 184 of the pressure insert 180 can be sized and shaped to slidably engage the upper planar alignment surfaces 117 with a slight friction fit, so as to maintain and hold the pressure insert 180 in an upward position within the central bore 115 until the upper body 120 had been coupled to the cylindrical base 140.

Moreover, as noted above, the ring retainer 150 can have a slit or slot 156 that allows for the temporary compression of the ring retainer 150 into a smaller diameter. In this way the spherical outer surface 154 of the slotted ring retainer 150 can be partially compressed into a smaller diameter as it is pushed downwards past the overtravel lip portion 142 during pre-assembly of the retainer sub-assembly 14 with the cylindrical base 140, and then released and allowed to expand back outwards to engage the seating surface 144 of the cylindrical base 140 with a friction fit. In one aspect the friction fit between the spherical outer surface 154 of the ring retainer 150 and the seating surface 144 of the cylindrical base 140, once the ring retainer 150 is positioned therein, can be strong enough to inhibit further movement of the ring retainer 150 and thereby maintain the center aperture 160 of the ring retainer 150 in co-axial alignment with the bottom opening 145 of the cylindrical base 140 during coupling of the cylindrical base 140 with the upper body 120, shipment and storage of the receiver sub-assembly 12, and eventually the mating of the receiver sub-assembly 12 with the capture portion 60 of a bone anchor 50. The upper body 120 (with enclosed pressure insert 180) can now be coupled to the cylindrical base 140 (with enclosed retainer sub-assembly 14) to complete the two-piece receiver 100 and at the same time form the multiplanar receiver sub-assembly 12.

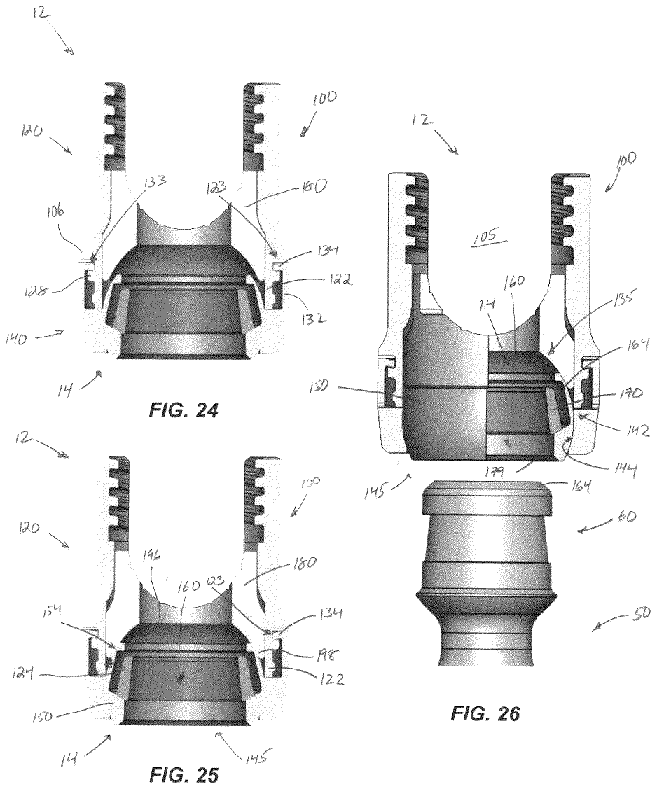

With reference to FIGS. 24-25, in one aspect the means or mechanism for coupling together the upper body 120 and the cylindrical base 140 can comprise the type of spring latch connection described above. For instance, the plurality of flexible curvate panels or spring tabs 132 having inwardly-projecting flanges or hook structures 134 at their upper ends can be formed into the annular upper structure 130 of the cylindrical base 140. The inwardly-projecting hook structures 134 can be sized and shaped to enter into the arcuate recesses 123 located between the upper surfaces of the arcuate ribs 128 and lower surfaces of the outwardly-flared portions 106 of the upper body 120.

During assembly of the upper body 120 and cylindrical base 140 to form the receiver sub-assembly 12, an initial engagement between the beveled upper edges 133 of the spring tabs 132 and the lower surfaces of the arcuate ribs 128 can cause the spring tabs 132 to flex outward so that the inward-facing tip surfaces of the hook structures 134 ride upwards along the outward-continue to move upwards until the hook structures 134 reach the level of the arcuate recesses 123, at which point the hook structures 134 of the spring tabs 132 can snap or latch into the annular recesses 123, thereby coupling together the upper body 120 and cylindrical base 140 of the two-piece receiver 100, as shown in FIG. 24. In one aspect the spring latch engagement between the cylindrical base 140 and upper body 120 of the two-piece receiver 100 can be configured so that the two pieces engage in a configuration that prevents rotation between the upper body 120 and the cylindrical base 140.

It is foreseen that other means, mechanisms or arrangements for coupling together the upper body 1120 and the cylindrical base 140 to form the receiver sub-assembly 12 are also possible and considered to fall within the scope of the present disclosure. In particular, it is contemplated that the arrangement of the flanges or hook structures and the recesses could be reversed, with the flanges or hook structures projecting radially outward from side curvate panels of the cylindrical base and with circumferential or arcuate recess(es) being formed into the inner cylindrical surfaces of the annular skirt of the upper body. It is further contemplated that the spring latch-type arrangement could also be flipped, with the plurality of flexible spring tabs extending downward from the annular lower end of the upper body and an inner cylindrical surfaces with the circumferential or arcuate recess(es) configured to receive the outwardly-projecting flanges being formed into the annular upper structure of the cylindrical base. Other types of connection mechanisms, such as threaded engagements, bayonet locking, camming, welding, and the like, are also contemplated and considered to fall within the scope of the present disclosure.

With reference to FIG. 25, after the inwardly-projecting hook structures 134 enter the annular recesses 123, the pressure insert 180 can then drop or be pushed back downwards toward the cylindrical base 140 and the retainer sub-assembly 14 until an annular outer portion of the concave lower surface 196 of the pressure insert 180 engages with the spherical outer surface 154 of the ring retainer 150. In doing so, the annular bottom edge portion 198 of the pressure insert 180 also enters into the space between the spherical outer surface 154 of the ring retainer 150 and the interior cylindrical surface 124 of the annular skirt 122. This action can further secure the ring retainer 150 of the retainer sub-assembly 14 within the internal cavity 135 of the two-piece receiver 100, with the center aperture 160 of the ring retainer 150 in co-axial alignment with the bottom opening 145 of the cylindrical base 140, as shown in the drawing figure. The receiver sub-assembly 12 is now complete and ready for coupling to the capture portion 60 of the bone anchor 50, which can be uploaded through the bottom opening 145 of the two-piece receiver 100, to complete the putting together of the multiplanar pivotal bone anchor assembly 10.

With reference now to FIG. 26, the ring retainer 150 of the retainer sub-assembly 14 can be frictionally secured within the internal cavity 135 of the two-piece receiver 100 by the overtravel lip portion 142 of the spherical seating surface 144, and with the center aperture 160 of the ring retainer 150 being maintained in alignment with the bottom opening 145 of the two-piece receiver 100, as described above. This can provide for the uploading of the capture portion 60 of the bone anchor 50 through the bottom opening 145 of the two-piece receiver 100, until the top edge or upper curvate section 64 of the capture portion 60 enters the center aperture 160 of the ring retainer 150 and freely travels upward to contact the bottom inner edge 179 of the capture ring 170 that, in turn, is supported within the internal recess 164 of the ring retainer 150.

As described with greater detail in co-owned U.S. Pat. No. 12,053,209, the capture portion 60 can continue to move upward through the center aperture 160 of the ring retainer 150 until the upper curvate section 64 passes completely through the ring retainer 150 and engages an annular inner portion of the concave lower surface 196 of the pressure insert 180. This contact occurs prior to the capture recess 70 of the capture portion 60 fully reaching the capture ring 170. The bone anchor 50 continues further upward, with the capture portion 60 pushing the pressure insert 180 back upwards within the central bore 115 of the two-piece receiver 100, until the upper outer slidable surface 68 of the capture portion 60 eventually slides upwardly out from under the capture ring 170, as shown in FIG. 27. This allows the capture ring 170 to snap into the horizontal capture recess 70 of the capture portion 60 and thereby couple the capture portion 60 directly to the retainer sub-assembly 14, and through the retainer sub-assembly 14 to the receiver sub-assembly 12.

With continued reference to FIG. 27, the outer curvate transition surfaces 188 of the pressure insert 180 can engage the complimentary inner curvate transition surfaces 118 of the opposite protrusions 116 of the two-piece receiver 100 simultaneous with or shortly after the snapping of the capture ring 170 into the capture recess 70, so as to prevent any further upward movement of the pressure insert 180 and the capture portion 60 relative to the two-piece receiver 100. This engagement between the outer curvate transition surfaces 188 and the inner curvate transition surfaces 118 can define the maximum push-through position of the bone anchor 50 relative to the receiver sub-assembly 12. Using these engagements between the pressure insert 180 and the two-piece receiver 100, and between the capture portion 60 and the pressure insert 180, as a hard stop for the upward motion of the bone anchor 50 relative to the receiver sub-assembly 12 can ensure that the upward passage of the bone anchor 50 does not push the retainer sub-assembly 14 up and out of its own captured position within the spherical seating surface 144 of the two-piece receiver 100. In addition, providing for the hard stop shortly after the capture ring 170 snaps into the horizontal capture recess 70 to complete the connection between the bone anchor 50 and the receiver sub-assembly 12 can provide a positive indication to the surgeon or medical professional that the coupling of the two components is now complete, while also limiting further error or complication if the surgeon pushes downward on the receiver sub-assembly 12 with too much force when attaching the receiver sub-assembly 12 to the capture portion 60 of a bone anchor 50 that has been implanted into the bone of a patient.

With reference to FIG. 28, once the capture ring 170 snaps into the horizontal capture recess 70 of the capture portion 60, the pressure insert 180 can again be downwardly displaced toward the retainer sub-assembly 14 until the annular outer portion of the concave lower surface 196 of the pressure insert 180 again engages with the spherical outer surface 154 of the ring retainer 150 simultaneous with the annular bottom edge portion 198 of the pressure insert 180 again entering into the space between the spherical outer surface 154 of the ring retainer 150 and the inner cylindrical surface of the annular skirt 122. This time, moreover, the concave lower surface 196 of the pressure insert 180 can also bear against the upper curvate section 64 of the bone anchor 50, so as to drive the capture portion 60 back downward through the center aperture of the ring retainer 150. As further disclosed in co-owned U.S. Pat. No. 12,053,209, the pressure insert 180 can drive the capture portion 60 back downward a short vertical distance until the upper and lower surfaces of the capture ring 170 become fulling engaged with their complementary surfaces on the capture portion 60 and the ring retainer 150, respectively, preventing further downward movement of the capture portion 60 relative to the ring retainer 150 and the two-piece receiver 100. It is also foreseen that the pressure insert 180 could be forced down with a tool from a first position to a second position so as to inhibit upward movement thereafter.

With continued reference FIG. 28, the retainer sub-assembly 14 is now coupled to the capture portion 60 of the bone anchor 50 to form the multiplanar pivotal bone anchor assembly 10, such that the retainer sub-assembly 14 and the capture portion 60 can articular together relative to the two-piece receiver 100 (and the pressure insert 180). Nevertheless, it will be appreciated that the fictional forces generated at the interface between the spherical outer surface 154 of the ring retainer 150 and the spherical seating surface 144 of the two-piece receiver 100, as discussed above, may also be strong enough to establish a non-floppy pre-lock friction fit that maintains the angular alignment between the two-piece receiver 100 and the articulating bone anchor 50/retainer sub-assembly 14 after their assembly together, while allowing for some repositioning or articulation by manually manipulating the two-piece receiver 100 so as to overcome the friction fit. It will also be appreciated that prior to locking with the elongate rod and closure, as described below, the internal connections between the capture portion 60, the capture ring 170, and the ring retainer 150 may be loose enough to allow the bone anchor 50 to freely rotate or spin relative to both the retainer sub-assembly 14 and the two-piece receiver 100.

As noted above, the outer surfaces of the insert body 190 adjacent the upwardly-extending side portions or insert arms 184 can include opposite flat or planar portions 186 that are configured to slidably engage with complementary opposing flat or planar portions 117 formed into the central bore 115, to establish and maintain alignment of the insert channel 185 with the open rod channel 105 of the upper body 120. In one aspect the outer surfaces of the insert and the inner surfaces of the central bore can also include features, such as protruding ridges or flanges that interact with one or more recessed grooves or notches, that can serve to establish and maintain a vertical position of the insert body within the central bore of the two-piece receiver until forcibly displaced in one direction or another, such as with tooling.

For example, in another exemplary embodiment of the pivotal bone anchor assembly (not shown), interacting protruding ridges and recesses or grooves may be used to hold the pressure insert within an upper portion of the annular lower end and central bore of the upper body prior to the cylindrical base and upper body being coupled together. After their connection to complete the two-piece receiver, a tool can then be used to press the pressure insert downwards until of the concave lower surface of the pressure insert engages with the spherical outer surface 154 of the ring retainer 150 to establish the shipping state condition, during which the protruding ridges may be moved downwards from an upper recess to a lower recess. The displacement of the pressure insert can be reversed during the uploading of the capture portion 60 of the bone anchor 50, in which case the protruding ridges may be moved back upwards from the lower recess to the upper recess as the pressure insert is driven upwards by the bone anchor 50 to the maximum push through of the capture portion 60 relative to the receiver sub-assembly. And finally, the tool can again be used to drive both the pressure insert and the capture portion 60 back downwards until the concave lower surface of the pressure insert again engages with the spherical outer surface 154 of the ring retainer 150.

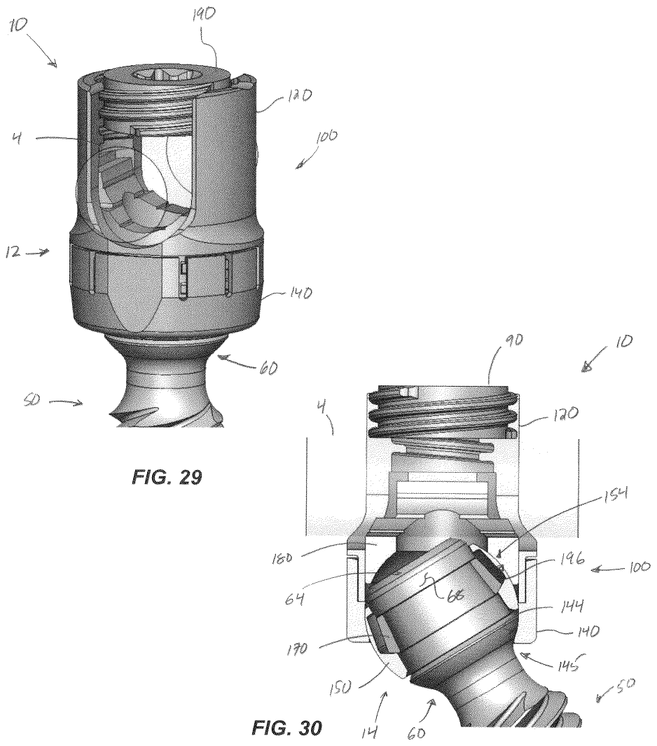

With reference to FIGS. 29-30, the elongate rod 4 and closure 90 can now be installed into the multiplanar bone anchor assembly 10 to complete the final assembly. As described with greater detail in co-owned U.S. Pat. No. 12,053,209, it is contemplated that the pressure provided by the closure 90 and elongate rod 4 can be used to establish a primary load path by driving an annular inner portion of the concave lower surface 196 of the pressure insert 180 downward against the upper curvate section 64 of the capture portion 60 of the bone anchor 50, which in turn can drive the capture ring 170 downward against the thick lower portion of the ring retainer 150. From there the primary load path can extend across the spherical seating surface 144 into the cylindrical base 140 of the multiplanar two-piece receiver 100, frictionally securing the position of both the bone anchor 50 and the retainer sub-assembly 14 relative to the two-piece receiver 100. The pressure provided by the closure 90 can also establish a secondary load path by driving an annular outer portion of the concave lower surface 196 of the pressure insert 180 against the spherical outer surface 154 of the ring retainer 150, which force is then transferred through the thin shell upper portion of the ring retainer 150 to the thick lower portion of the ring retainer 150, and from there to the seating surface 144 and cylindrical base 140 of the multiplanar two-piece receiver 100.

Furthermore, the portion of the pressure provided by the closure 90 that follows the secondary load path can also cause the concave lower surface 196 of the pressure insert 180 clamp against the spherical outer surface 154 of the ring retainer 150 with enough pressure to close the upper portion of the slit or slot 156 and to deform the thin upper portion of the ring retainer 150 into an additional frictional engagement with the upper outer slidable surface 68 of the capture portion 60. Accordingly, the multiple fictional engagements established between the concave lower surface 196 of the pressure insert 180, the upper curvate section 64 and upper outer slidable surface 68 of the capture portion 60, the spherical outer surface 154 of the ring retainer 150, and the spherical seating surface 144 of the two-piece receiver 100 can establish a final locked engagement that prevents further articulation and rotation of the two-piece receiver 100 relative to the bone anchor 50.

Through continuous research and development of new bone anchor designs, it has been determined that the significant pressures or loads being transferred through the various components of the multiplanar bone anchor assembly 10 can be high enough to approach or even exceed the local yield strength of the metal material(s) that form the individual components. In one aspect these slight local inelastic deformations may be useful by causing the separate internal components of the bone anchor assembly to compress and bind together to form a more solidly locked assembly or unit upon final locking with the elongate rod and closure. Nevertheless, in most situations it is necessary to maintain a comfortable margin between the pressure loads carried by the component and the yield strength of the material in order to account for variations in tolerances and occasional inconsistencies in imperfect manufacturing processes, thereby ensuring that the components and assemblies do not fail during implantation and use and are reliably strong over time.

One complicating factor in maintaining these load-bearing margins in the different components is the desire to scale downward the designs and functionalities of adult-sized bone anchor assemblies that are generally sized for use with elongate rods or longitudinal connecting members having rod diameters of about 6.50 mm to about 5.50 or 5.0 mm. In particular, it may be desirable to scale the adult-sized designs downward so as to be used with small stature/pediatric or cervical applications having rod diameters of 5.0 to about 2.5 mm. With the smaller rod sizes, it is preferrable that the overall size of the bone anchor assembly, and in particular the size of the central bore and bottom opening of the receiver and the outer diameter of the closure, are also reduced. However, it is notable that while the internal components can be reduced in size to fit within the smaller receiver, these reductions can also result in additional limitations with assembling both the internal components of the receiver sub-assembly and the capture portion of the bone anchor into the smaller receiver.

One design factor that can remain substantially constant is the pressure load provided by the closure that is required to lock a pivotal bone anchor assembly, both large and small, into its final locked configuration. In designs where the pressure load remains substantially constant while the size of the components in the load path(s) supporting these pressure loads are reduced, the local reduction in cross-sectional area can quickly result in increased stress levels that reach or exceed the yield strength of the material. Consequently, the different internal components are often simplified and/or removed altogether in bone anchor assemblies designed for small stature/pediatric or cervical applications, which can also strip away the improved functionalities provided by those components in adult-sized bone anchor assemblies.

Through continued modeling, analysis, and testing, it has been discovered that one solution for overcoming these challenges is to separate a solid integral receiver into a two-piece receiver, as described above. Once separated, the upper portion 120 of the two-piece receiver 100 that defines the channel 105 and central bore 115 can be modified to receive a reduced-size elongate rod, closure, and at least the upper portion of the pressure insert 180 that also receives the reduced-sized elongate rod. At the same time, the size of the cylindrical base 140 or lower portion of the two-piece receiver 100 that defines the internal cavity 135 and bottom opening 145 can be generally maintained to allow the lower ‘retaining’ components of the bone anchor assembly 10, that are defined by or received within the internal cavity, to preserve their adult-or near adult-sized dimensions, together with their larger cross-sectional areas and acceptable load-bearing margins.

As noted above, this novel approach to re-designing the receiver can also facilitate the pre-assembly of the separate internal components into the two-piece receiver to form the receiver sub-assembly, with the internal components (e.g., the pressure insert and the retainer sub-assembly) being loadable into the upper or lower pieces of the two-piece receiver prior to their coupling together, as described above, rather than being downloaded through the central bore or uploaded through the bottom opening. It will be appreciated that with smaller-sized bone anchor assemblies, this approach can also preserve the use of the larger, higher-functional internal components that are configured to interface with the universal shank heads described above, so as to provide the bone anchor assembly or spinal fixation system with the same modular capabilities as their adult-sized counterparts. In other words, in addition to maintaining their greater load-bearing capabilities, the smaller-sized two-piece receiver sub-assemblies can also preserve the higher-level functionalities, including but not limited to multiplanar, monoplanar, monoaxial, favored-angle, and independent lock capabilities, etc., that are configured for coupling with an uploadable universal shank head, as provided by their comparable adult-sized bone anchor assemblies and spinal fixation systems.

In addition to the above, it will be appreciated the bone anchor assembly or spinal fixation system described above may be considered “modular” in the sense that any particular type of receiver sub-assembly, in the shipping state condition, can be coupled with any one of a variety of bone anchors 50 having anchor portions of different size, length, type, and/or thread patterns, but with all of the bone anchors 50 having the same universal capture structure, such as capture structure 60, at their upper ends. Furthermore, it will also be appreciated that a receiver sub-assembly 12 in the shipping state condition can be assembled with the bone anchor 50 at the hospital or surgery center either before insertion into the patient, or after the threaded shank or bone anchor 50 has been inserted into the patient (such as directly by a surgeon or with robotic assistance). The different techniques or approaches for the insertion and assembly of the modular parts of the bone anchor assembly can be described as ex-vivo and in-vivo or in-situ, respectively.

It is further noted that the multiplanar two-piece receiver 100 can provide additional representative methods of assembling the bone anchor together with the components of the receiver sub-assembly 12. For example, in one representative method embodiment the capture portion 60 of the bone anchor 50 could first be pre-assembled with the retainer sub-assembly 14, and followed by downloading the anchor portion of the bone anchor 50 through the bottom opening 145 of the cylindrical base 140 until the ring retainer 150 of the retainer sub-subassembly 23 becomes positioned within the spherical seating surface 144, after which the upper body 120 of the two-piece receiver 100 is coupled to the cylindrical base 140. In yet another representative method embodiment, the retainer sub-subassembly 14 and the cylindrical base 140 can be pre-assembled separately from the pressure insert 180 and the upper body 120, followed by the capture portion 60 being uploaded through the bottom opening 145 and into the retainer sub-assembly 14, after which the upper body 120 of the two-piece receiver 100 can then be coupled to the cylindrical base 140.

### Monoaxial Bone Anchor Assembly

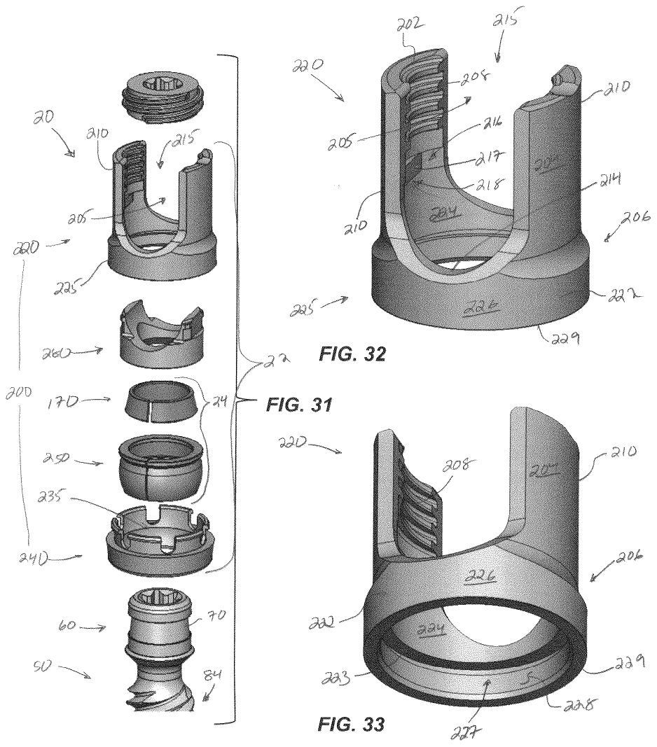

Referring now to FIG. 31, illustrated therein is an exploded perspective view of one representative embodiment of the non-pivotal, relatively “fixed”, or monoaxial bone anchor assembly 20 that is configured to substantially limit pivotal motion of the bone anchor relative to the receiver sub-assembly (or vice versa) while still providing for rotational motion around a 360-degree range. The monoaxial bone anchor assembly 20 can include the same bone anchor 50 or bone screw described above, having a universal capture portion 60 or shank head and an anchor portion 84 opposite the capture portion 60 for securement or attachment to the bone of a patient. Similar to the multiplanar assembly 10 discussed above, the monoaxial bone anchor assembly 20 can also include a two-piece receiver 200 or housing having a cylindrical base 240 defining the lower portion of an internal cavity 235 that is configured to receive the capture portion 60 of the bone anchor 50, and an upper body 220 that includes a pair of upright arms 210 integrally formed with and extending upward from an annular lower end 225 to define an open rod channel 205 configured to receive an elongate rod (not shown). The annular lower end 225 can further define the upper portion of the internal cavity 235, and the annular lower end 225 and upright arms 210 can together define a central bore 215 that extends upward from the internal cavity 235 through the rod channel 205 to the tops of the upright arms 210, and which is centered about the vertical centerline axis 201 of the monoaxial receiver.

The two-piece receiver 200 of FIG. 31 can be pivotably secured to the capture portion 60 of the bone anchor 50 with a number of separate internal components that have been pre-assembled into the internal cavity 235 and the central bore 215 to form a monoaxial receiver sub-assembly 22. These internal components can include a monoaxial ring retainer 250 with the separate open capture ring 170 secured therein (which ring retainer 250 and capture ring 170 together define a non-pivoting retainer sub-assembly 24) and a pressure insert 280. After the elongate rod has been positioned within the lower portion of the rod channel 205, a closure 90 can be threadably or otherwise secured into an upper portion of the rod channel 205/central bore 215 to apply pressure to an upper surface of the elongate rod, such as by direct contact therebetween, thereby locking the elongate rod and the monoaxial bone anchor assembly 20 together into a final locked position.

The primary difference between the pivotal multiplanar bone anchor assembly 10 previously described and the non-pivotal monoaxial bone anchor assembly 20 of FIG. 31 can be the replacement of the pivotal ring retainer, having continuous spherical outer surfaces, with the monoaxial ring retainer 250 having a circumferential ridge protruding outwardly from an upper portion thereof and having an annular planar top surface that is engageable by an annular planar bottom surface of the monoaxial pressure insert 280. The remainder of the components forming the monoaxial bone anchor assembly 20, such as the universal shank 50, the capture ring 170, and the closure 90, can be the same as or substantially similar to those already described, so as to more completely provide a modular spinal fixation system with an array of receiver sub-assemblies having all of the attendant benefits thereof.

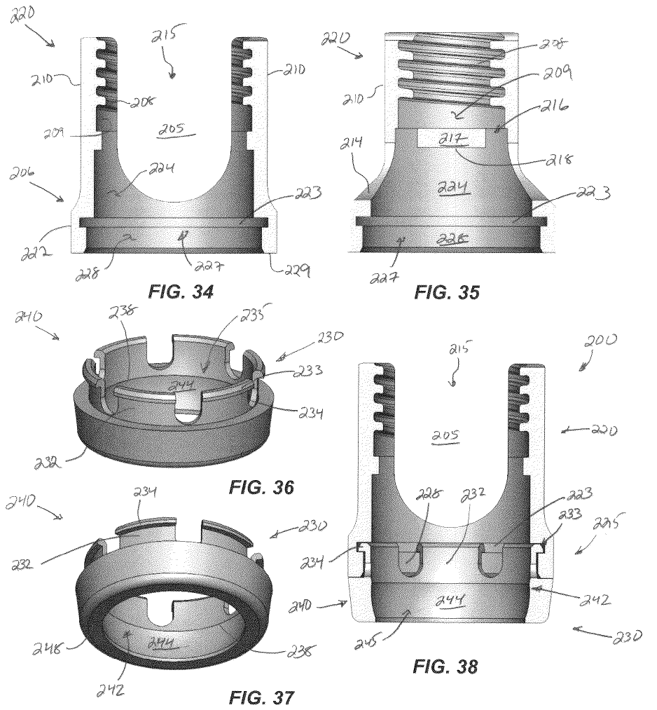

With reference to FIGS. 32-38, the two-piece receiver 200 of the monoaxial bone anchor assembly 20 can have much the same construction and features of the pivotal receiver embodiment 100 described above, with the exception of the construction of the spring latch mechanism formed into the annular lower end 225 of the upper body 220 and the annular upper structure 230 of the cylindrical base 240, as well as the inwardly-extending protrusions 216 defining the upper planar alignment surfaces 217 that can be shorter in length and with planar, downwardly-facing lower arcuate stop surfaces 218 rather than a lower curvate transition surfaces 118. Many remaining structural elements of the upper body 220 of the two-piece receiver 200, including the upright arms 210 forming the rod channel 205, the discontinuous guide and advancement structure 208 formed into the upper portion of the central bore 215, the curvate side outer surfaces 204 of the upright arms 210, the lower, outwardly-flared portions 206 located proximate the U-shaped saddle surfaces 214 that define the lower portion of the rod channel 205, the circular lower edge 229 defining the lower opening 227, and the like, can be the same as or substantially similar to the structural elements of the multiplanar two-piece receiver described above.

Likewise, the cylindrical base 240 of the two-piece receiver 200 can also define the lower portion of the internal cavity 235 that communicates with the bottom surface 248 of the cylindrical base 240 through a bottom opening 245, with the lower portion of the internal cavity 235 including a spherical seating surface 244 that is sized and shaped to closely receive the spherical outer surface portion 254 of the ring retainer 250 of the retainer sub-assembly 24. In one aspect the seating surface 244 can also include an overtravel lip portion 242 that is configured to extend above a hemisphere plane of the spherical outer surface 254 of the ring retainer 250, but in the monoaxial embodiment to a circumferential internal edge 238 that defines the upper border of the seating surface 244, rather than to the internal ledge of the previous embodiment. It will be appreciated, nevertheless, that with the monoaxial embodiment of the bone anchor assembly 20 it may not be necessary for the seating surface 244 of the cylindrical base 240 to be spherical or curvate so as to provide for pivotal motion between the receiver 200 and the bone anchor 50, and that other shapes or geometries for the seating surface are also possible and considered to fall within the scope of the present disclosure.

As shown in FIG. 32-35, the construction of the spring latch mechanism for coupling the upper body 220 with the cylindrical base 240 of the two-piece receiver 200 can include the annular lower end 225 of the upper body 220 having a downwardly-extending annular skirt 222 with a bottom edge surface 229, and a lower inner cylindrical surface 228. The upper body 220 can also include an upper inner cylindrical surface 224 that extends upwardly into the open rod channel 205 until it reaches the runout groove 209 of the discontinuous guide and advancement structure 208 that is formed into upper portions of the internal surfaces of the upright arms 210, and an annular recess 223 located between the lower and upper inner cylindrical surfaces. In one aspect the lower inner cylindrical surface 228 can have a diameter that is greater than the diameter of the upper inner cylindrical surface 224 so as to accommodate the annular upper structure 130 of the cylindrical base 140.

With reference to FIGS. 36-37, the cylindrical base 240 can include a plurality of flexible spring tabs 232, with outwardly-projecting protuberances or flanges 234 at their upper ends, which define the annular upper structure 230. The outwardly-projecting flanges 234 can be sized and shaped to enter into the annular recess 223 located between the lower inner cylindrical surface 228 and the upper inner cylindrical surface 224 of the annular skirt 222 shown in FIGS. 32-35. Different aspects the spring latch engagement between the cylindrical base 240 and the upper body 220 of the two-piece receiver 200 can be configured so that the two pieces engage in a configuration that can prevent rotation, or alternatively can provide for rotation, between the upper body 220 and the cylindrical base 240.

During assembly of the upper body 220 and cylindrical base 240 to form the monoaxial two-piece receiver 200, an initial engagement between the beveled top surfaces 233 of the spring tabs 230 and the circular lower edge 229 of the annular skirt 222, can cause the spring tabs 232 to flex inward so that the outer tip surfaces of the outwardly-projecting flanges 234 ride upwards along the lower inner cylindrical surface 228 of the annular skirt 222. The spring tabs 230 can continue to move upward until the outwardly-projecting flanges 234 reach the level of the annular recess 223, at which point the flanges 234 of the spring tabs 232 can snap or latch into the annular recess 223, thereby coupling together the upper body 220 and cylindrical base 240 of the two-piece receiver 100, as shown in FIG. 38.

It will be appreciated that the spring latch mechanism of the monoaxial two-piece receiver 200 is a substantial reversal of the spring latch mechanism of the multiplanar two-piece receiver 100, in that the flanges or hook structures 234 project radially outward from the spring tabs 232 of the cylindrical base 240 and with the circumferential recess 223 being formed into the inner cylindrical surfaces 228 of the annular skirt 222 of the upper body 220. It will also be appreciated that the different types of spring latch mechanisms can be interchangeable, and that the spring latch mechanism of the multiplanar embodiment could be replaced with that of the monoaxial embodiment. In this configuration (not shown), the lower cylindrical outer surface and the annular bottom edge portion of the pressure insert can enter into the space between the spherical outer surface of the ring retainer and the interior surfaces of the spring tabs upon completion of the receiver sub-assembly. It is foreseen that this action would serve to further secure the upper body of the two-piece receiver to the cylindrical base by blocking the backsides of the spring tabs and preventing the hook structures from backing out or disengaging from the circumferential recess(es).

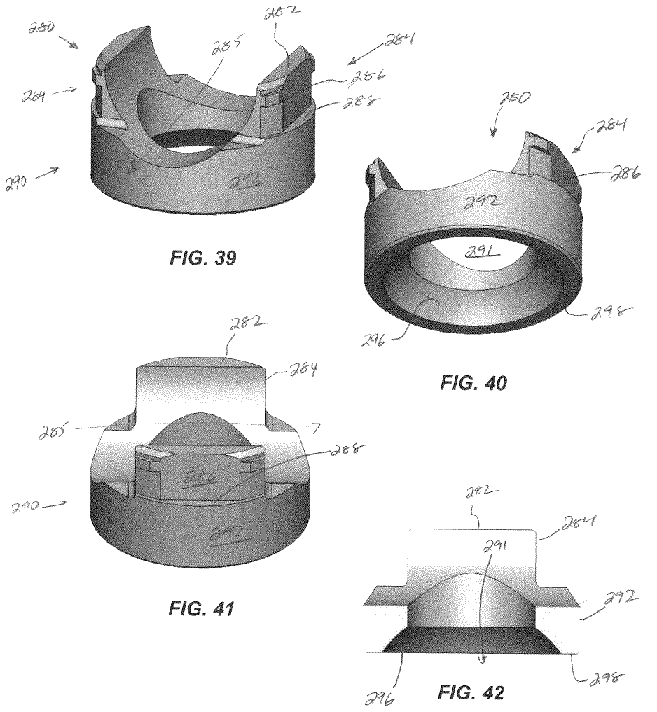

With reference to FIGS. 39-42, the monoaxial pressure insert 280 can also be substantially similar to the multiplanar pressure insert described above, in that the pressure insert 280 can also comprise a generally-cylindrical insert body 290 with a center aperture 291 that is alignable with the vertical centerline axis of the two-piece receiver 200 and having a linear upper groove or insert channel 285 formed therein that is configured for engagement by the lower surface portion of the elongate rod. The insert body 290 of the pressure insert 280 can also include a lower cylindrical outer surface 292 extending downward to an annular bottom surface 298, and which is configured for slidable engagement within the upper inner cylindrical surface 224 of the upper body 220 of the two-piece receiver 200. As shown in the drawings, the insert body 290 can further include a downward-facing concave lower surface 296 that is configured to engage the spherical or curvate upper portion 64 of the capture portion 60 upon the capture portion's uploading through the bottom opening 245 of the two-piece receiver 200.

The monoaxial pressure insert 280 can differ from the multiplanar embodiment in that the insert body 290 can be shorter in the axial direction, with the height of both the lower cylindrical outer surface 192 and the downward-facing concave lower surface 296 being reduced so that the annular bottom surface 298, configured to engage the annular top surface of the ring retainer 250, is greater in radial width. In addition, the outer surfaces of the two upwardly-extending side portions or insert arms 284 can modified to include opposite flat or planar outer surfaces 286 extending downward from the top surfaces 282 to planar, upwardly-facing arcuate engagement surfaces 288 that are configured to abut the complimentary downwardly-facing lower arcuate stop surfaces 218 formed into the internal surfaces of the upright arms 210 of the upper body 220 when the pressure insert 280 is in its most upwardly-advanced position.

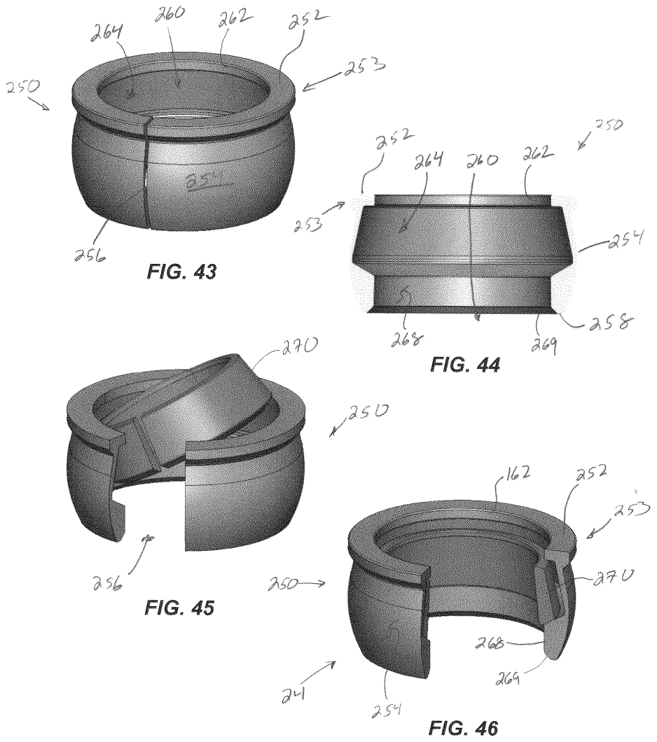

With reference to FIGS. 43-44, the monoaxial ring retainer 250 can also be similar to the multiplanar ring retainer described above, in that the ring retainer 250 can also comprise an O-ring body having a curvate outer surface, such as spherical outer surface 254 extending upward from a bottom annular or edge surface 258, and a center aperture 260 defined by inner slidable surfaces 262, 268 configured to loosely slidably engage with the outer slidable surfaces 68, 74 of the capture portion 60 of the bone anchor 50. The ring retainer 250 can also include an internal recess 264 that extends into and circumferentially around a mid-portion of the center aperture 260, as well as a lower beveled surface 269 extending between the lower inner slidable surface 268 and the bottom annular or edge surface 258 that can be complementary with the beveled lip surface 75 of the capture portion 60 of the shank 50. The ring retainer 250 can also include a slit or slot 256 extending through the thickness of the O-ring body, from the center aperture 260 through to the outer surface 254, to form an open ring retainer that is expandable so as to allow the capture ring 270 to be preloaded or positioned within the internal recess 264, and which is also compressible so as to allow the ring retainer 250 to be pressed into the spherical seating surface 244 of the cavity 135 of the monoaxial two-piece receiver 200 so as to provide for a pre-lock friction fit and alignment stability for the retainer sub-assembly 24 with respect to the two-piece receiver 200.

The monoaxial ring retainer 250 can differ from the multiplanar embodiment in that the O-ring body can include a circumferential ridge structure 253 at the upper end of the ring retainer 280 that extends radially outward beyond the spherical outer surface 254. The circumferential ridge structure 253 can serve to increase the radial width of the top annular surface 262 that is configured for engagement with the annular bottom surface 298 of the pressure insert 280, as described in more detail below.

As previously noted, it is understood that the open capture ring 270 shown in FIGS. 45-46 can be the same as or substantially similar to the open capture ring 170 that is positionable within the multiplanar ring retainer 150 of the multiplanar bone anchor assembly 10 discussed above, respectively, and can be configured with the same structure and to perform the same functions during the assembly and operation of the monoaxial retainer sub-assembly 24 as with the multiplanar retainer sub-assembly 14.

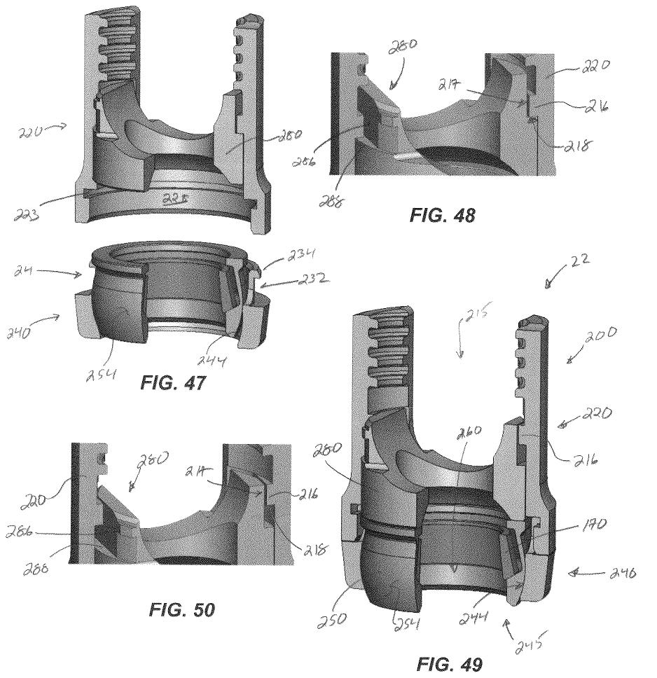

To begin the pre-assembly of the monoaxial receiver sub-assembly 22, the capture ring 170 can first be installed or positioned into the internal recess 264 of the ring retainer 250 to form the retainer sub-assembly 24, as shown in FIGS. 45-46. With reference now to FIG. 47, the spherical outer surface 254 of the slotted ring retainer 250 can then be partially compressed into a smaller diameter as it is pushed downwards past the overtravel lip portion 242 during pre-assembly of the retainer sub-assembly 24 with the cylindrical base 240, and then released and allowed to expand back outwards to engage the seating surface 244 of the cylindrical base 240 with a friction fit.

With continued reference to FIG. 47, the pressure insert 280 can also be uploaded into the central bore 215 of the upper body 220 of the two-piece receiver 200 through the lower opening 227, with the lower cylindrical outer surface 292 of the pressure insert entering into slidable engagement with the upper inner cylindrical surface 224 of the upper body 220 and the insert channel 285 being generally aligned with the rod channel 205. The pressure insert 280 can then be moved upwardly within the central bore 215 until the opposite planar outer surfaces 286 on the arms 284 of the pressure insert 280 slidably engage with the upper planar alignment surfaces 217, so as to establish and maintain the insert channel 285 in alignment with the open rod channel 205 of the upper body 220. With continued upward movement of the pressure insert 280, the planar arcuate engagement surfaces 288 of the pressure insert 280 can eventually abut the planar arcuate stop surfaces 218 of the protrusions 216, as shown in FIG. 48, so as to provide a hard stop that restricts further upward movement of the pressure insert 280 relative to the upper body 220.

The upper body 220 of the monoaxial two-piece receiver 200 (with enclosed pressure insert 280) can now be coupled to the cylindrical base 240 (with enclosed retainer sub-assembly 24) to complete the two-piece receiver 200 and at the same time form the monoaxial receiver sub-assembly 22. As described above, an initial engagement between the beveled top surfaces 233 of the spring tabs 230 and the circular lower edge 229 of the annular skirt 222 can cause the spring tabs 232 to flex inward so that the outer tip surfaces of the outwardly-projecting flanges 234 ride upwards along the lower inner cylindrical surface 228 of the annular skirt 222. The spring tabs 230 can continue to move upward until the outwardly-projecting flanges 234 reach the level of the annular recess 223, at which point the flanges 234 of the spring tabs 232 can snap or latch into the annular recess 223, thereby coupling together the upper body 220 and cylindrical base 240 of the two-piece receiver 200. After the outwardly-projecting flanges 234 enter the annular recess 223, the pressure insert 280 can then drop or be pushed back downwards toward the cylindrical base 240 and the retainer sub-assembly 24 until the annular bottom surface 298 of the pressure insert 280 engages with the top annular surface 262 of the ring retainer 250, as shown in FIGS. 49-50. The downward displacement of the pressure insert 280 into engagement with the top annular surface 262 of the ring retainer 250 can operate to complete the pre-assembly of the monoaxial receiver sub-assembly 22.

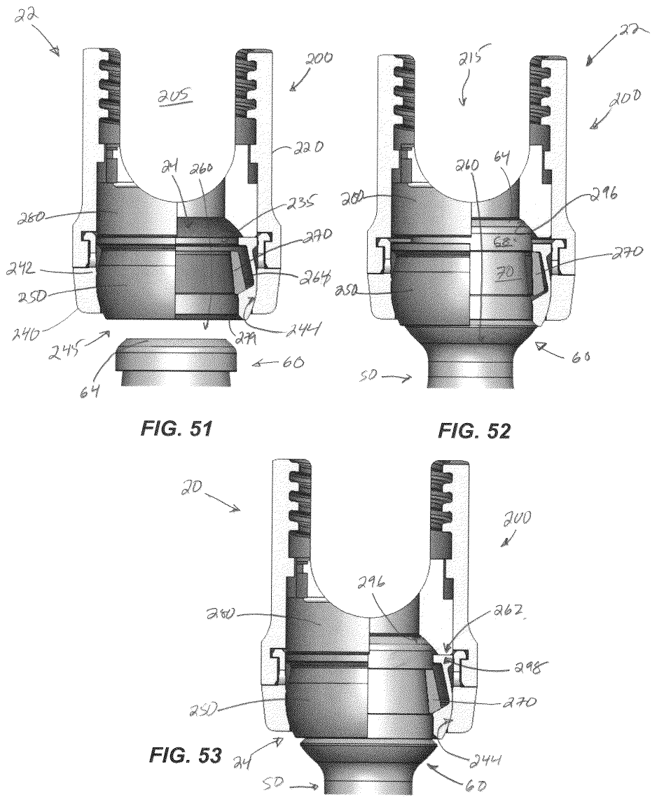

The capture portion 60 of the bone anchor 50 can now be uploaded through the bottom opening 245 of the two-piece receiver 200 and into the retainer sub-assembly 24 to complete the putting together of the monoaxial bone anchor assembly 20. With reference to FIG. 51, the ring retainer 250 of the retainer sub-assembly 24 can be frictionally secured within the internal cavity 235 of the two-piece receiver 200 by the overtravel lip portion 242 of the spherical seating surface 244, and with the center aperture 260 of the ring retainer 250 being maintained in alignment with the bottom opening 245, as described above. The capture portion 60 of the bone anchor 50 can then be uploaded through the bottom opening 245 until the top edge or upper curvate section 64 of the capture portion 60 enters the center aperture 260 of the ring retainer 250 and freely travels upward to contact the bottom inner edge 279 of the capture ring 170 that, in turn, is supported within the internal recess 264 of the ring retainer 250.

As described with greater detail in co-owned U.S. Pat. No. 12,053,209, the capture portion 60 can continue to move upward through the center aperture 260 of the monoaxial ring retainer 250 until the upper curvate section 64 passes completely through the ring retainer 250 and engages an annular inner portion of the concave lower surface 296 of the pressure insert 280 to push the pressure insert 280 back upwards within the central bore 215 of the two-piece receiver 200, as shown in FIG. 52. Eventually the upper outer slidable surface 68 of the capture portion 60 slides upwardly out from under the capture ring 270, allowing the capture ring 270 to snap into the horizontal capture recess 70 of the capture portion 60 and thereby couple the capture portion 60 directly to the retainer sub-assembly 24, and through the retainer sub-assembly 24 to the receiver sub-assembly 22.

Once the capture ring 270 snaps into the horizontal capture recess 70 of the capture portion 60, the pressure insert 280 can again be downwardly displaced toward the retainer sub-assembly 24, with the concave lower surface 296 of the pressure insert 280 bearing against the upper curvate section 64 of the bone anchor 50. This can serve to drive the capture portion 60 back downward through the center aperture 260 of the ring retainer 250 a short vertical distance until the upper and lower surfaces of the capture ring 170 become fulling engaged with their complementary surfaces on the capture portion 60 and the ring retainer 250, respectively, simultaneous with the annular bottom surface 298 of the pressure insert 280 again engaging with the top annular surface 262 of the ring retainer 250, as shown in FIG. 53.

With continued reference FIG. 53, the retainer sub-assembly 24 is now coupled to the capture portion 60 of the bone anchor 50 to form the monoaxial bone anchor assembly 20, such that the internal connections between the capture portion 60, the capture ring 170, and the ring retainer 250 can allow the bone anchor 50 to freely rotate or spin relative to the two-piece receiver 200 while preventing articulation, or pivotal motion, of the monoaxial retainer sub-assembly 24 and the capture portion 60 relative to the two-piece receiver 200

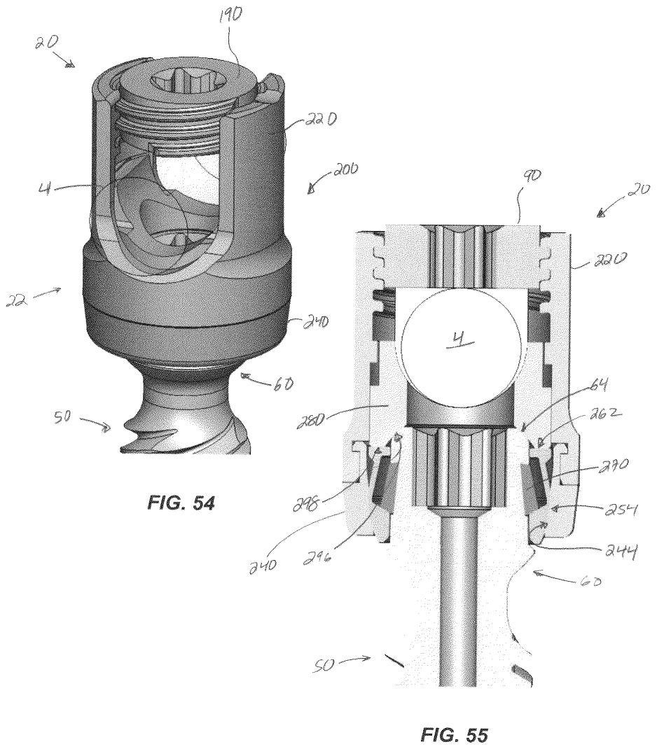

With reference to FIGS. 54-55, the elongate rod 4 and closure 90 can now be installed into the monaxial bone anchor assembly 20 to complete the final assembly. As described with greater detail in co-owned U.S. Pat. No. 12,053,209, it is contemplated that the pressure provided by the closure 90 and elongate rod 4 can be used to establish both a primary load path and a secondary load path through the bone anchor assembly 20, respectively, by driving the concave lower surface 296 of the pressure insert 280 downward against the upper curvate section 64 of the capture portion 60 of the bone anchor 50 simultaneous with driving the annular bottom surface 298 of the pressure insert 280 against the top annular surface 262 of the ring retainer 250. Accordingly, the multiple fictional engagements established between the concave lower surface 296 and annular bottom surface 298 of the pressure insert 280, the upper curvate section 64 of the capture portion 60, the top annular surface 262 of the ring retainer 250, and the spherical seating surface 244 of the two-piece receiver 200 can establish a final locked engagement that prevents further rotation of the two-piece receiver 200 relative to the bone anchor 50.

As indicated above, the invention has been described herein in terms of preferred embodiments and methodologies considered by the inventor to represent the best mode of carrying out the invention. It will be understood by the skilled artisan, however, that a wide range of additions, deletions, and modifications, both subtle and gross, may be made to the illustrated representative embodiments of the bone anchor assemblies without departing from the spirit and scope of the invention. As such, these and other revisions might be made by those of skill in the art without departing from the spirit and scope of the invention that is constrained only by the following claims.

What is claimed is:

1. A pivotal bone anchor assembly for securing an elongate rod to a bone of a patient with a closure, the pivotal bone anchor assembly comprising:
a receiver comprising:
an upper body comprising an annular lower end defining an upper portion of an internal cavity, a pair of upright arms extending upward from the annular lower end to define an open channel configured to receive the elongate rod, and a central bore centered about a vertical centerline axis and extending downward from tops of the upright arms through the open channel to the upper portion of the internal cavity, the annular lower end including a first connection structure extending downwardly opposite the pair of upright arms; and
a cylindrical base defining a lower portion of the internal cavity in communication with a bottom of the cylindrical base through a bottom opening, the cylindrical base including a spherical seating surface adjacent the bottom opening and a second connection structure extending above the spherical seating surface, the second connection structure configured for coupling with the first connection structure to secure the cylindrical base to the upper body and complete the internal cavity;
a bone anchor comprising a capture portion and an anchor portion opposite the capture portion configured for attachment to the bone;
a retainer sub-assembly comprising:
a ring retainer having a center aperture, an internal recess, and a spherical outer surface slidably engageable with the spherical seating surface of the cylindrical base; and
a separate open capture ring secured within the internal recess of the ring retainer and having a center opening alignable with the center aperture of the ring retainer; and
a pressure insert comprising an insert channel alignable with the channel of the receiver and configured to receive the elongate rod, and a downward-facing concave lower surface configured to engage an upper surface of the capture portion of the bone anchor,
wherein the retainer sub-assembly is positionable into the cylindrical base of the receiver and the pressure insert is positionable into the upper body of the receiver prior to the cylindrical base and the upper body being coupled together to complete the internal cavity, and
wherein after the cylindrical base and the upper body are coupled together to complete the internal cavity, the capture portion of the bone anchor is configured for uploading into the completed internal cavity through the bottom opening and for being captured therein by the retainer sub-assembly.

2. The pivotal bone anchor assembly of claim 1,
wherein the first connection structure comprises a downwardly-extending annular skirt having an inward-facing annular recess formed therein, and the second connection structure comprises a plurality of flexible spring tabs having outwardly-projecting flanges at their upper ends, and
wherein the plurality of flexible spring tabs are configured to flex inward as the second connection structure is uploaded into the first connection structure, until the outwardly-projecting flanges of the cylindrical base snap into the annular recess of the upper body to secure the cylindrical base to the upper body and complete the internal cavity.

3. The pivotal bone anchor assembly of claim 1, wherein pressure insert includes a lower skirt portion with an outer cylindrical surface configured for slidable engagement the plurality of flexible spring tabs to prevent the outwardly-projecting flanges of the plurality of flexible spring tabs from disengaging from the annular recess of the upper body.

4. The pivotal bone anchor assembly of claim 1, wherein the completed internal cavity is laterally-enclosed around the circumference thereof.

5. The pivotal bone anchor assembly of claim 1, wherein the capture portion further comprises an upper slidable outer surface, a lower slidable outer surface, and a capture recess between the upper slidable outer surface and the lower slidable outer surface configured to receive the open capture ring of the retainer sub-assembly.

6. The pivotal bone anchor assembly of claim 5, wherein the upper slidable outer surface and the lower slidable outer surface are frustoconical.

7. The pivotal bone anchor assembly of claim 1, wherein the spherical seating surface extends upward from the bottom opening to a circumferential ledge that defines an upper end of an overtravel lip structure formed into the lower portion of the internal cavity above an equator line of the spherical seating surface.

8. The pivotal bone anchor assembly of claim 7, wherein the ring retainer is an open ring retainer having a slot or slot extending entirely through the thickness thereof and from a top surface to a bottom surface.

9. The pivotal bone anchor assembly of claim 8, wherein the spherical outer surface of the ring retainer is configured for compressive frictional engagement by the spherical seating surface of the cylindrical base to provide a non-floppy pivotal friction fit to inhibit pivotal motion between the bone anchor and the receiver prior to downloading the elongate rod into the open channel and locking the pivotal bone anchor assembly with the closure.

10. The pivotal bone anchor assembly of claim 1 and further comprising the elongate rod and the closure, wherein the closure is configured for positioning entirely within the central bore of the receiver above the elongate rod and in engagement with a mating structure formed into the central bore to apply a downward pressure to a top of the elongate rod, so as to secure the elongate rod to the bone of the patient.

11. A pivotal bone anchor assembly for securing an elongate rod to a bone of a patient with a closure, the pivotal bone anchor assembly comprising:
a receiver comprising:
an upper body comprising an annular lower end defining an upper portion of an internal cavity, a pair of upright arms extending upward from the annular lower end to define an open channel configured to receive the elongate rod, and a central bore centered about a vertical centerline axis and extending downward from tops of the upright arms through the open channel to the upper portion of the internal cavity, the annular lower end including a first connection structure extending downwardly opposite the pair of upright arms; and
a cylindrical base defining a lower portion of the internal cavity in communication with a bottom of the cylindrical base through a bottom opening, the cylindrical base including a seating surface adjacent the bottom opening and a second connection structure extending above the spherical seating surface, the second connection structure configured for coupling with the first connection structure to secure the cylindrical base to the upper body and complete the internal cavity;
a bone anchor comprising a capture portion and an anchor portion opposite the capture portion configured for attachment to the bone;
a retaining structure comprising a center aperture configured to receive the capture portion of the bone anchor and an outer surface engageable with the seating surface of the cylindrical base; and
a pressure insert comprising an insert channel alignable with the channel of the receiver and configured to receive the elongate rod, and a downward-facing concave lower surface configured to engage an upper surface of the capture portion of the bone anchor,
wherein the retaining structure is positionable into the cylindrical base of the receiver and the pressure insert is positionable into the upper body of the receiver prior to the cylindrical base and the upper body being coupled together to complete the internal cavity, and
wherein after the cylindrical base and the upper body are coupled together to complete the internal cavity, the capture portion of the bone anchor is configured for uploading into the completed internal cavity through the bottom opening and for being captured therein by the retaining structure.

12. The pivotal bone anchor assembly of claim 11,
wherein the seating surface of the cylindrical base is a spherical seating surface and the outer surface of the retainer structure is a spherical outer surface slidably engageable with the spherical seating surface of the cylindrical base, and
wherein the retaining structure is configured to articulate with the bone anchor upon the capture of the capture portion with the completed internal cavity prior to locking the pivotal bone anchor assembly with the closure.

13. The pivotal bone anchor assembly of claim 12, wherein the spherical seating surface extends upward from the bottom opening to a circumferential ledge that defines an upper end of an overtravel lip structure formed into the lower portion of the internal cavity above an equator line of the spherical seating surface.

14. The pivotal bone anchor assembly of claim 13, wherein the retaining structure is an open ring retainer having a slot or slot extending entirely through the thickness thereof and from a top surface to a bottom surface.

15. The pivotal bone anchor assembly of claim 14, wherein the spherical outer surface of the open ring retainer is configured for compressive frictional engagement by the spherical seating surface of the cylindrical base to provide a non-floppy pivotal friction fit to inhibit pivotal motion between the bone anchor and the receiver prior to downloading the elongate rod into the open channel and locking the pivotal bone anchor assembly with the closure.

16. A method of assembling a pivotal bone anchor assembly configured for securing an elongate rod to a bone of a patient, the method comprising:
positioning a pressure insert into an upper portion of an internal cavity as defined by an upper body of a receiver,
the upper body of the receiver comprising an annular lower end defining the upper portion of the internal cavity, a pair of upright arms extending upward from the annular lower end to define an open channel configured to receive the elongate rod, and a central bore centered about a vertical centerline axis and extending downward from tops of the upright arms through the open channel to the upper portion of the internal cavity, the annular lower end including a first connection structure extending downwardly opposite the pair of upright arms, and
the pressure insert comprising an insert channel alignable with the channel of the receiver and configured to receive the elongate rod, and a downward-facing concave lower surface configured to engage an upper surface of the capture portion of the bone anchor;
positioning a retainer sub-assembly into a lower portion of the internal cavity as defined by a cylindrical base of the receiver,
the cylindrical base of the receiver defining the lower portion of the internal cavity in communication with a bottom of the cylindrical base through a bottom opening, the cylindrical base including a spherical seating surface adjacent the bottom opening and a second connection structure extending above the spherical seating surface, the second connection structure configured for coupling with the first connection structure to secure the cylindrical base to the upper body and complete the internal cavity, and
the retainer sub-assembly comprising:
a ring retainer having a center aperture, an internal recess, and a spherical outer surface slidably engageable with the spherical seating surface of the cylindrical base; and
a separate open capture ring secured within the internal recess of the ring retainer and having a center opening alignable with the center aperture of the ring retainer;
coupling together the cylindrical base and the upper body of the receiver to complete the internal cavity; and
uploading the capture portion of a bone anchor into the completed internal cavity through the bottom opening until the capture portion is captured therein by the retainer sub-assembly, the bone anchor comprising the capture portion and an anchor portion opposite the capture portion configured for attachment to the bone.

17. The method of claim 16,
wherein the first connection structure comprises a downwardly-extending annular skirt having an inward-facing annular recess formed therein, and the second connection structure comprises a plurality of flexible spring tabs having outwardly-projecting flanges at their upper ends, and
wherein the plurality of flexible spring tabs are configured to flex inward as the second connection structure is uploaded into the first connection structure, until the outwardly-projecting flanges of the cylindrical base snap into the annular recess of the upper body to secure the cylindrical base to the upper body and complete the internal cavity.

18. The method of claim 17, wherein pressure insert includes a lower skirt portion with an outer cylindrical surface configured for slidable engagement the plurality of flexible spring tabs to prevent the outwardly-projecting flanges of the plurality of flexible spring tabs from disengaging from the annular recess of the upper body.

19. A method of assembling a pivotal bone anchor assembly configured for securing an elongate rod to a bone of a patient, the method comprising:
positioning a pressure insert into an upper portion of an internal cavity as defined by an upper body of a receiver,
the upper body of the receiver comprising an annular lower end defining the upper portion of the internal cavity, a pair of upright arms extending upward from the annular lower end to define an open channel configured to receive the elongate rod, and a central bore centered about a vertical centerline axis and extending downward from tops of the upright arms through the open channel to the upper portion of the internal cavity, the annular lower end including a first connection structure extending downwardly opposite the pair of upright arms, and
the pressure insert comprising an insert channel alignable with the channel of the receiver and configured to receive the elongate rod, and a downward-facing concave lower surface configured to engage an upper surface of the capture portion of the bone anchor;
positioning a retaining structure into a lower portion of the internal cavity as defined by a cylindrical base of the receiver,
the cylindrical base of the receiver defining the lower portion of the internal cavity in communication with a bottom of the cylindrical base through a bottom opening, the cylindrical base including a seating surface adjacent the bottom opening and a second connection structure extending above the spherical seating surface, the second connection structure configured for coupling with the first connection structure to secure the cylindrical base to the upper body and complete the internal cavity, and
the retaining structure comprising a center aperture configured to receive the capture portion of the bone anchor and an outer surface engageable with the seating surface of the cylindrical base:
coupling together the cylindrical base and the upper body of the receiver to complete the internal cavity; and
uploading the capture portion of a bone anchor into the completed internal cavity through the bottom opening until the capture portion is captured therein by the retaining s structure, the bone anchor comprising the capture portion and an anchor portion opposite the capture portion configured for attachment to the bone.
